(19) United States
(12) Patent Application Publication
(10) Pub. No.: US 2009/0136570 A1
(43) Pub. Date: May 28, 2009

(54) Taste-Masked Tablets and Granules

(76) Inventors: Bhagwant Rege, Collegeville, PA (US); Kimberly Kaighn, Glen Mills, PA (US); Soumojeet Ghosh, Lansdale, PA (US); Laman Alani, Foster City, CA (US)

Correspondence Address: MERCK AND CO., INC, P O BOX 2000, RAHWAY, NJ, 07065-0907, US

(21) Appl. No.: 12/087,827

(22) PCT Filed: Jan. 16, 2007
(86) PCT No.: PCT/US2007/001027

§ 371 Date: Jul. 16, 2008

Related U.S. Application Data
(60) Provisional application No. 60/760,537, filed on Jan. 20, 2006.

(51) Int. Cl. A61K 9/20; A61K 31/513; A61K 31/519; A61P 31/18; A61K 31/506

(52) U.S. Cl. 424/465; 514/269; 514/259.4; 514/215

(57) ABSTRACT

Orally administered, taste-masked tablets and granules contain (a) a hydroxypyrimidinone carboxamide, a hydroxy-tetrahydropyridopyrimidinone carboxamide, or a related carboxamide compound, or a pharmaceutically acceptable salt thereof, (b) a taste-masking polymer, (c) a superdisintegrant, and optionally other excipients. The carboxamide compound is an HIV integrase inhibitor, and the tablets and granules are suitable for use in the inhibition of HIV integrase, the treatment or prophylaxis of HIV infection, and the treatment or prophylaxis or delay in the onset of AIDS.

### FIELD OF THE INVENTION

The present invention is directed to orally administered, taste-masked tablets and granules that contain a hydroxypyrimidinone carboxamide, a hydroxy-tetrahydropyridopyrimidinone carboxamide, or a related carboxamide compound, a taste-masking polymer, and a superdisintegrant. The carboxamide is an HIV integrase inhibitor and the tablets and granules are useful in the treatment of HIV infection or AIDS.

### BACKGROUND OF THE INVENTION

Certain hydroxypyrimidinone carboxamides such as those disclosed in US 2005/0025774 and certain hydroxy-tetrahydropyridopyrimidinone carboxamides and related carboxamides such as those disclosed in WO 2004/058756 are HIV integrase inhibitors useful for the treatment of HIV infection and AIDS. Although these carboxamides can have an unpleasant, bitter, or otherwise disagreeable taste when administered orally, the taste problem can be circumvented by administering the carboxamides in the form of a capsule or a coated tablet that dissolves or disintegrates in the stomach after being swallowed. Unfortunately certain patient populations (e.g., children and the elderly) can have difficulty swallowing such capsules and tablets. Other dosage forms such as orally disintegrating tablets (i.e., tablets that quickly dissolve in the mouth without water), chewable tablets and liquid suspensions are in principle easier to swallow, but the disagreeable taste of the carboxamide can itself lead to swallowing difficulties with these dosage forms and/or cause patients to avoid taking the drug altogether thereby leading to low compliance. Accordingly, there is a need for oral dosage forms containing the carboxamide integrase inhibitors that are both palatable and easy to swallow and thus suitable for use among pediatric and geriatric patient populations.

The following references are of interest as background:

Ishikawa et al., Chem. Pharm. Bull. 1999, 47 (10): 1451-1454 discloses the preparation of tablets containing a taste-masked drug that can rapidly disintegrate in saliva. It is disclosed that an ethanol-based gel of EUDRAGIT E-100 containing a bitter tasting drug (pirenzepine HCl or oxybutynin HCl) was extruded; the ethanol was removed from the extrudate by overnight evaporation to provide a string-shaped solid; the solid was crushed to provide taste-masked granules; the granules were mixed with crystalline cellulose, low-substituted hydroxypropylcellulose, and magnesium stearate; and the mixture compressed into tablets. These tablets were found to disintegrate rapidly in saliva and have no bitter taste in tests conducted with healthy volunteers.

U.S. Pat. No. 6,221,402 B1 discloses a rapidly releasing and taste-masking pharmaceutical dosage form that has a core containing the active pharmaceutical ingredient, low-substituted hydroxypropyl cellulose, and microcrystalline cellulose; an inner coating layer formed on the core containing a water soluble polymer; and an outer coating layer formed on the inner coating layer containing a saliva-insoluble polymer. It is disclosed that the outer coating layer has a taste-masking effect and suitable saliva-insoluble polymers include aminoalkyl methacrylate copolymers such as EUDRAGIT E.

US 2003/0181501 A1 discloses a process for preparing an intraorally disintegrating valdecoxib composition wherein the process comprises providing particulate valdecoxib; adding a dissolution retardant (e.g., EUDRAGIT E PO) to the particulate valdecoxib to form a composite; admixing the composite with an excipient that exhibits rapid oral dissolution to provide a tableting blend; granulating the particulate valdecoxib, the composite, or the tableting blend; and compressing the tableting blend to form a tablet. In one embodiment, the composition is a taste-masking fast-melt tablet.

Taste masked pharmaceutical dosage forms are also described in WO 2004/022037 A1 and WO 2004/066984 A2.

### SUMMARY OF THE INVENTION

The present invention is directed to taste-masked pharmaceutical oral dosage forms containing a hydroxypyrimidinone carboxamide, a tetrahydropyridopyrimidinone carboxamide, or a hexahydropyrimidoazepine carboxamide, or a pharmaceutically acceptable salt thereof. More particularly, the present invention includes a pharmaceutical oral dosage form which is a tablet or granules, wherein the dosage form comprises:

(a) an effective amount of a carboxamide compound or a pharmaceutically acceptable salt thereof, wherein the carboxamide compound is (i) a 1-alkyl-5-hydroxy-6-oxo-1,6-dihydropyrimidine-4-carboxamide compound, (ii) a 3-hydroxy-4-oxo-6,7,8,9-tetrahydro-4H-pyrido[1,2-a]pyrimidine-2-carboxamide compound, or (iii) a 3-hydroxy-4-oxo-4,6,7,8,9,10-hexahydropyrimido[1,2-a]azepine-2-carboxamide compound;

(b) a taste-masking polymer; and

(c) a superdisintegrant.

The dosage form optionally contains additional excipients as described in more detail below.

The taste-masked oral dosage form of the present invention can be a tablet, and the taste-masked tablet can be an orally disintegrating tablet (“ODT”) that can quickly dissolve in the mouth without water and thus can be taken at any time anywhere. The taste-masked tablet can alternatively be a chewable tablet that can administered and chewed in the mouth with or without water. The taste-masked oral dosage form of the present invention can also be granules which can be suspended in a liquid or given with a soft food (e.g., apple sauce). The taste-masked tablets and granules of the present invention are palatable, convenient, and easy to take and can lead to better patient compliance. They are particularly suitable for pediatric and geriatric patients who have difficulty taking capsules, tablets, or other solid dosage forms that do not dissolve or disintegrate until after they are swallowed.

Although the taste-masked tablets and granules encompassed by the present invention are particularly suitable for use by pediatric and geriatric populations, they are also suitable for use by patients (e.g., young adults) who have no difficulty taking orally administered solid dosage forms that are designed to maintain their structural integrity prior to swallowing.

Various embodiments, aspects and features of the present invention are either further described in or will be apparent from the ensuing description, examples and appended claims.

### DETAILED DESCRIPTION OF THE INVENTION

The taste-masked pharmaceutical oral dosage forms of the present invention include an effective amount of a carboxamide compound as described above in the Summary of the Invention and as described in more detail below (see, e.g., the description below of compounds of Formula I). These carboxamide compounds are HIV integrase inhibitors. Representative carboxamide compounds have been tested in an integrase inhibition assay in which strand transfer is catalyzed by recombinant integrase, and have been found to be active inhibitors of HIV integrase. Integrase inhibition activity can be determined, for example, using the assay described in flazuda et al., J. Virol. 1997, 71: 7005-7011. Representative compounds have also been found to be active in an assay for the inhibition of acute HIV infection of T-lymphoid cells conducted in accordance with Vacca et al., Proc. Natl. Acad. Sci. USA 1994, 91: 4096-4100. Further description of representative carboxamide compounds, methods for their preparation, and assays for measuring their integrase inhibition activity and their inhibition of HIV replication can be found in WO 03/035077, US 2005/0025774, and WO 2004/058756, the disclosures of which are herein incorporated by reference in their entireties.

As used herein, the term “dosage form” or “formulation” is intended to encompass an orally administered, solid dosage product comprising the specified ingredients, as well as any product which results, directly or indirectly, from combining the specified ingredients to form the product.

The term “effective amount” as used herein means that amount of a carboxamide compound as described herein (or that amount of another active agent) that elicits the biological or medicinal response in a tissue, system, animal or human that is being sought by a researcher, veterinarian, medical doctor or other clinician. The effective amount can be a “therapeutically effective amount” for the alleviation of the symptoms of the disease or condition being treated. The effective amount can also be a “prophylactically effective amount” for prophylaxis of the symptoms of the disease or condition being prevented. With respect to a carboxamide compound as described herein, the term also refers to the amount of the compound sufficient to inhibit HIV integrase and thereby elicit the response being sought (i.e., an “inhibition effective amount”). The carboxamide compound can be employed in the oral dosage form of the invention in an amount, for example, in a range of from about 1 to about 75 wt. %, is typically employed in an amount in a range of from about 1 to about 50 wt. % (e.g., from about 3 to about 30 wt. %), and is more typically employed in an amount in a range of from about 5 to about 25 wt. %, on a free phenol basis.

Unless expressly stated to the contrary, all ranges cited herein are inclusive. For example, a weight percent range of from about 1 to about 50 wt. % means the weight percent can be about 1 wt. %, or about 50 wt. %, or any value in between.

The oral dosage forms of the present invention can be described as palatable. The term “palatable” means herein that the dosage form has an acceptable taste in the mouth. A palatable dosage form can be but is not necessarily pleasant or agreeable in taste; it is instead a form which is not unpleasant or disagreeable. For example, a dosage form having a “chalky” taste is considered herein as palatable, even though the dosage form might be considered as not pleasant in taste

An embodiment of the present invention (alternatively referred herein to as Embodiment E1) is a pharmaceutical oral dosage form as originally defined above (i.e., as defined in the Summary of the Invention), wherein the oral dosage form is granules prepared by a process which comprises dry blending the carboxamide compound or its pharmaceutically acceptable salt, the taste-masking polymer, the superdisintegrant, and optionally one or more other excipients; and granulating the dry blend (e.g., dry granulating by rolling the dry blend to form a compact, and then milling the compact to form granules).

As used herein, the term “dry blending” means mixing the components together in the absence of a liquid medium; i.e., no liquid (e.g, no water or alcohol) is employed to dissolve, suspend, and/or disperse the blend components. The components are typically blended until an intimate, relatively homogeneous (i.e., no layers or coatings) admixture is obtained. It is understood that the ingredients which are dry blended can be added to the blender concurrently or at different times in any order and a particular ingredient can be added all at once or in separate portions at different times during the dry blending step.

Another embodiment of the present invention (Embodiment E2) is a pharmaceutical oral dosage form as originally defined above, wherein the oral dosage form is a tablet (e.g., a chewable tablet) prepared by a process which comprises dry blending the carboxamide compound or the pharmaceutically acceptable salt thereof, the taste-masking polymer, the superdisintegrant, and optionally one or more other excipients; granulating the dry blend to provide granules (e.g., dry granulating by rolling the dry blend to form a compact, and then milling the compact to form granules); and compressing the granules. In an aspect of Embodiment E2, the tablet resulting from the compression of the granules is a chewable tablet having a hardness of at least about 6 kiloponds (e.g., from about 6 to about 10 kiloponds). In another aspect of Embodiment E2, the tablet resulting from the compression of the granules is a chewable tablet having a hardness in a range of from about 6 to about 9 kiloponds (e.g., from about 7 to about 8 kiloponds).

The hardness of a tablet (i.e., the force required to crush the tablet) can be measured using a Key Model HT-300 or 14T-500 Hardness Tester (available from Key International, Englishtown, N.J.). The hardness is suitably expressed in units of kiloponds, wherein 1 kilopond=9.81 Newton.

Another embodiment of the present invention (Embodiment E3) is a pharmaceutical oral dosage form as originally defined above, wherein the oral dosage form is a tablet (e.g., an ODT) prepared by a process which comprises dry blending the carboxamide compound or the pharmaceutically acceptable salt thereof and the taste-masking polymer, and optionally one or more other excipients; granulating the dry blend to provide granules (e.g., dry granulating by rolling the dry blend to form a compact, and then milling the compact to form granules); mixing the granules with the superdisintegrant and optionally one or more other extragranular excipients; and compressing the mixture. In an aspect of Embodiment E3, the tablet resulting from the compression of the granules is an ODT having a hardness of less than about 6 kiloponds (e.g., from about 2 to less than 6 kiloponds). In another aspect of Embodiment E3, the tablet resulting from the compression of the granules is an ODT having a hardness in a range of from about 2 to about 5 kiloponds (e.g., from about 2 to about 4 kiloponds).

Masking the unpleasant (e.g., bitter) taste of drugs is often accomplished by coating the drug with a taste-masking polymer such as by applying (e.g., via spraying or a fluidized bed) a suspension of the taste-masking polymer on the drug particles per se or on granules containing the drug particles, and then drying the polymer-coated drug particles or drug-containing granules. It is believed that the oral dosage forms of the invention can achieve effective taste masking by dry blending the taste masking polymer with the carboxamide compound in lieu of a coating step. Avoiding a coating step is desirable, not only because it eliminates a step from the overall formulation process but also because the application of the coating is not straightforward, involving the selection and optimization of a suitable coating suspension and the selection, control, and optimization of a suitable coating process. Fluid bed coating processes, for example, can be difficult to operate reliably, having numerous process parameters to be controlled and optimized.

The palatability of the dry-blend based dosage forms of the invention can be determined, for example, by having human subjects to whom the dosage form is administered complete a taste questionnaire in which the subject is asked, inter alia, to judge the sweetness (from much too sweet to needs a lot more sweetness), bitterness (from not at all bitter to extremely bitter), mouth taste (from very pleasant to very unpleasant), and after taste (from very pleasant to very unpleasant) immediately following administration.

Another embodiment of the present invention (Embodiment E4) is a pharmaceutical oral dosage form as originally defined above or as defined in any preceding embodiment, wherein

(a) the carboxamide compound or its pharmaceutically acceptable salt is employed in an amount in a range of from about 1 to about 75 wt. % on a free phenol basis;

(b) the taste-masking polymer is employed in an amount in a range of from about 1 to about 40 wt. %; and

(c) the superdisintegrant is employed in an amount in a range of from about 1 to about 25 wt. %.

The weight percent of the active ingredient (i.e., the carboxamide compound) is expressed in terms of the free base even if the compound is employed in the form of a salt, and the weight percents of the other components are each based upon the total weight of the dosage form including the weight of the salt when the carboxamide compound is employed in the form of a salt. In the preceding embodiment, when the components (a), (b), and (c) are employed in amounts the sum of which is less than 100% (including the weight of the carboxamide salt when a salt is employed), other excipients such as those described below (e.g., a filler) are included in the dosage form.

It is understood that the weight percentages of the ingredients recited in any composition or formulation set forth herein (such as Embodiment E4 above) cannot total more than 100 wt. % It is also understood that when the total weight of the recited ingredients typically can have a total of less than 100 wt. %, in which case other unspecified excipients are present in the dosage form.

Another embodiment of the present invention (Embodiment E5) is a pharmaceutical oral dosage form as originally defined above, which further comprises: (d) a compression aid; (e) a water soluble filler; and (f) a lubricant. In an aspect of this embodiment, the oral dosage form is granules prepared by a process which comprises dry blending the ingredients and then granulating the dry blend (e.g., dry granulating by rolling the dry blend to form a compact, and then milling the compact to form granules). In another aspect of this embodiment, the oral dosage form is a tablet (e.g., a chewable tablet) prepared by dry blending the carboxamide compound or its pharmaceutically acceptable salt, the taste-masking polymer, the superdisintegrant, the compression aid, the water soluble filler, and the lubricant; granulating the dry-blend (e.g., dry granulating by rolling the dry blend to form a compact, and then milling the compact to form granules); and compressing the granules to obtain the tablet. In still another aspect of this embodiment, the oral dosage form is a tablet (e.g., an ODT) prepared by a process which comprises dry blending the carboxamide compound or its pharmaceutically acceptable salt, the taste-masking polymer, the compression aid, and a first portion or all of the lubricant; granulating the dry blend to provide granules; mixing the granules with the water soluble filler, the superdisintegrant, and the remaining portion (if any) of the lubricant; and compressing the mixture. In the dry blending step of this aspect, for example, the carboxamide compound or its pharmaceutically acceptable salt, the taste-masking polymer and the compression aid can first be added to the blender and blended until well mixed, and then the lubricant can be added and the admixture blended until the lubricant is well mixed therewith. In the step of mixing the granules in this aspect, for example, some of the remaining portion of the lubricant can first be mixed with the granules and then the other ingredients (i.e., the water soluble filler, the superdisintegrant, and the rest of the remaining portion of the lubricant) can be mixed with the lubricated granules.

The lubricant employed in Embodiment E5 and the aspects thereof can be a combination of two lubricants. By the term “combination” is meant that the two lubricants can be mixed together and employed as a mixture, or they can be used separately. In particular, when the lubricant is employed in more than one step for making a tablet, one of the lubricants of the combination can be employed in one step (e.g., granulating the dry blend in the above-described aspect) and the other lubricant of the combination can be employed in a separate step (e.g., mixing with the granules in the above-described aspect).

In the foregoing aspects of Embodiment E5, the ingredients which are dry blended can be added to the blender concurrently or at different times in any order. Furthermore, each particular ingredient can be added all at once or added in two or more portions at the same or different times in the dry blending step.

The taste-masking polymer employed in the pharmaceutical oral dosage form of the present invention can be any polymer which masks or hides the taste of the carboxamide compound active ingredient which can have a bitter and/or otherwise unpleasant taste. Suitable taste masking polymers include a cellulose polymer, a vinyl carboxylate-alkylene glycol copolymer, an acrylic polymer, a methacrylic polymer, or an acrylic-methacrylic copolymer. As used herein, an acrylic polymer is a polymer which is substantially composed of units of acrylic acid and/or one or more acrylic acid derivatives [e.g., alkyl esters, substituted alkyl esters (e.g., aminoalkyl esters), primary amides, secondary amides, and tertiary amides]. A methacrylic polymer is a polymer which is substantially composed of units of methacrylic acid and/or one or more methacrylic acid derivatives [e.g., alkyl esters, substituted alkyl esters (e.g., aminoalkyl esters), primary amides, secondary amides, and tertiary amides]. The term “substantially” in this context means that at least about 80%, preferably at least about 90% (e.g., from about 95% to about 100%), more preferably at least about 95% (e.g., from about 98% to 100%), and most preferably 99-100% of the units are the units specified.

A class of suitable taste-masking polymers is hydroxypropylmethylcellulose (EPMC), hydroxypropylcellulose (HPC), ethylcellulose, cellulose acetate, a vinyl acetate-ethylene glycol copolymer, or a methacrylic copolymer. A preferred taste-masking polymer is an aminoalkyl methacrylate copolymer such as Eudragit® E 100 or Eudragit® E PO.

The taste masking polymer can be employed in the oral dosage form of the invention in any amount which effectively masks the unpleasant taste of the dosage form. The taste-masking polymer can be employed, for example, in an amount in a range of from about 1 to about 40 wt. %, is typically employed in an amount in a range of from about 1 to about 25 wt. %, and is more typically employed in an amount in a range of from about 2 to about 15 wt.% The pharmaceutical oral dosage form of the invention also contains a superdisintegrant, which is a substance employed to facilitate breakup or disintegration of the formulation in the mouth after administration. Suitable superdisintegrants include croscarmellose sodium, crospovidone, and sodium starch glycolate. The superdisintegrant can be employed in the oral dosage form of the invention in any amount which effectively facilitates disintegration of the dosage form in the mouth. The superdisintegrant can be employed, for example, in an amount in a range of from about 1 to about 25 wt.%, is typically employed in an amount in a range of from about 1 to about 15 wt.%, and is more typically employed in an amount in a range of from about 1 to about 5 wt.% The pharmaceutical oral dosage form of the invention can also contain a compression aid which is a substance that enhances the compactibility and compressibility of the composition in the formation of granules and tablets. Substances suitable for use as a compression aid include lactose, sucrose, anhydrous dibasic calcium phosphate, dibasic calcium phosphate dihydrate, tribasic calcium phosphate, calcium sulfate, carboxymethylcellulose calcium, microcrystalline cellulose, and powdered cellulose. A preferred compression aid is microcrystalline cellulose. Suitable forms of microcrystalline cellulose for use in the pharmaceutical oral dosage forms of the invention include, but are not limited to, the materials sold as AVICEL PH-101, AVICEL PH-102, AVICEL PH-103, and AVICEL PH-105 (all of which are available from FMC Biopolymer), and mixtures thereof. Another suitable form is a silicified microcrystalline cellulose such as Prosolv® (available from JRS Pharma, Patterson, N.Y.). Thus, for example, the microcrystalline cellulose employed in a tablet or granules of the invention can be AVICEL PH-102. The compression aid can be employed in the oral dosage form of the invention in any amount which is effective in enhancing the compactibility of the dosage form. The compression aid can be employed, for example, in an amount in a range of from about 5 to about 70 wt. %, is typically employed in an amount in a range of from about 5 to about 50 wt.% and is more typically employed in an amount in a range of from about 5 to about 25 wt.%

The pharmaceutical oral dosage form of the invention can also contain a water soluble filler which is a substance that provides bulk to the dosage form and can also dissolve relatively quickly in the mouth following administration. Suitable water soluble fillers include sugars such as mannitol, glucose, dextrose, or sucrose. Mannitol is a preferred water soluble filler. The water soluble polymer can be employed in the oral dosage form of the invention in any amount which provides suitable bulk to the dosage form. The water soluble polymer can be employed, for example, in an amount in a range of from about 5 to about 75 wt. %, is typically employed in an amount in a range of from about 10 to about 65 wt. %, and is more typically employed in an amount in a range of from about 30 to about 60 wt. %

The pharmaceutical oral dosage form of the invention can also contain a lubricant which can have one or more functions depending upon the dosage form of the composition. The lubricant can, for example, prevent adhesion of compressed tablets to the compression equipment and/or it can improve the flow of granules prepared via granulation of the composition. Suitable lubricants include calcium stearate, glyceryl monostearate, glyceryl palmitostearate, hydrogenated castor oil, hydrogenated vegetable oil, light mineral oil, magnesium stearate, mineral oil, polyethylene glycol, stearic acid, talc, zinc stearate, and sodium stearyl fumarate. Preferred lubricants are magnesium stearate, Na steayl fumarate, and a combination thereof. The lubricant can be employed in the oral dosage form of the invention in any amount which is effective in sufficiently lubricating the dosage form. The lubricant or combination of lubricants can be employed, for example, in an amount in a range of from about 0.1 to about 10 wt. %, is typically employed in an amount in a range of from about 0.1 to about 8 wt. %, and is more typically employed in an amount in a range of from about 0.5 to about 3 wt. %

Another embodiment of the present invention (Embodiment E6) is a pharmaceutical oral dosage form as originally defined above or as defined in any preceding embodiment, which further comprises (g) a sweetening agent.

The sweetening agent, alone or in combination with a flavoring agent (see below), can enhance the palatability of the tablet or granules to the intended consumer upon disintegration and/or chewing in the mouth following oral administration. Sweetening agents suitable for use in the present invention include sodium saccharin, cyclamate, aspartame, acsulfame potassium, sucralose, and combinations thereof. The term “combination” of sweetening agents has a meaning analogous to that set forth above with respect to lubricants; i.e., it means that two or more sweetening agents are present in the oral dosage form and can be employed in the same step or different steps of the preparation of the dosage form. If employed in the same preparative step, the sweetening agents can first be mixed together and the mixture employed in the preparative step or the agents can be employed separately either at the same time or at different times in the preparative step.

A sweetening agent is an optional component of the oral dosage form suitably employed in an amount effective to provide a palatable sweetness to the tablet as it disintegrates and/or is chewed during oral administration. The sweetening agent, or combination of sweetening agents, can be employed, for example, in an amount in a range of from about 0.1 to about 10 wt. %, is typically employed in an amount in a range of from about 0.1 to about 5 wt. %, and is more typically employed in an amount in a range of from about 0.1 to about 3 wt. % of the total weight of the dosage form (e.g., total weight of the tablet).

Another embodiment of the present invention (Embodiment E7) is a pharmaceutical oral dosage form as originally defined above or as defined in any preceding embodiment, which further comprises (h) a taste modifier.

The taste modifier, alone or in combination with a flavoring agent and/or a sweetening agent, can enhance the palatability of the tablet to the intended consumer when it disintegrates and/or is chewed during oral administration. Taste modifiers suitable for use in the present invention include monoammonium glycyrrhizinate (e.g., Magnasweet®, available from MAFCO, Camden, N.J.), sorbitol, maltose, maltodextrin, dextrose, fructose, and combinations thereof. The term “combination” as applied to taste modifiers has a meaning analogous to that set forth above for a combination of sweetening agents. A taste modifier is an optional component of the tablet formulation suitably employed in an amount effective to provide a palatable taste to the tablet as it disintegrates and/or is chewed during oral administration. The taste modifier, or combination of taste modifiers, can be employed, for example, in an amount in a range of from about 0.1 to about 3 wt. %, is typically employed in an amount in a range of from about 0.1 to about 2 wt. %, and is more typically employed in an amount in a range of from about 0.1 to about 1 wt. % of the total weight of the dosage form.

Taste modifiers can be and typically are also sweet, but are considered different from sweetening agents. A sweetening agent is included in the oral dosage form of the invention to provide an immediate and acute sweetness to the mouth following administration, whereas a taste modifier is included to provide a lingering sweetness and freshness to the mouth; i.e., the taste modifier is included to provide a lingering pleasant feeling in the mouth.

Another embodiment of the present invention (Embodiment E8) is a pharmaceutical oral dosage form as originally defined above or as defined in any preceding embodiment, which further comprises (i) a flavoring agent.

The flavoring agent is a substance that enhances the palatability of the dosage form (i.e., tablet or granules) to the intended consumer when it disintegrates and/or is chewed during oral administration. Flavoring agents suitable for use in the present invention include natural flavors (e.g., natural fruit flavors), artificial flavors (e.g., artificial fruit flavors), and combinations thereof. The term “combination” as applied to flavoring agents has a meaning analogous to that set forth above for a combination of sweetening agents. Natural and artificial flavors include mint (such as peppermint or spearmint), menthol, cinnamon, vanilla, artificial vanilla, chocolate, artificial chocolate and bubblegum. Natural and artificial fruit flavors include banana, cherry, grape, orange, strawberry, melon and lemon. A flavoring agent is an optional component of the oral dosage form and is suitably employed in an amount effective to provide a palatable flavor to the tablet as it disintegrates and/or is chewed during oral administration. The flavoring agent, or a combination of agents, is suitably employed in an amount in a range of from about 0.1 to about 10 wt. %, is typically employed in an amount in a range of from about 0.1 to about 5 wt. %, and is more typically employed in an amount in a range of from about 0.1 to about 3 wt. % of the total weight of the dosage form.

The use of a sweetening agent and/or a taste modifier and/or a flavoring agent in a pharmaceutical oral dosage form of the invention is optional. On the other hand, preferred oral dosage forms of the invention include a sweetening agent and a taste modifier, and more preferred forms include a sweetening agent, a taste modifier, and a flavoring agent.

Other components of the pharmaceutical tablet of the invention can provide a sweetening and/or taste modifying effect. For example, certain water soluble fillers (e.g., mannitol) and certain compression aids (e.g., lactose and sucrose) can also provide sweetness and/or taste modification. Accordingly, when the employment of a sweetening agent, a taste modifier and/or a flavoring agent in the dosage form is contemplated, the use of these other components can minimize the amount of or preclude employment of the sweetening agent, the taste modifier and/or the flavoring agent while still maintaining palatability. Suitable adjustment of the kinds and amounts of tablet components to impart the desired palatability can be done without undue experimentation by a person of ordinary skill in the art.

Another embodiment of the present invention (Embodiment E9) is a pharmaceutical oral dosage form set forth above in Embodiment E5, wherein:

(a) the carboxamide compound or its pharmaceutically acceptable salt is employed in an amount in a range of from about 1 to about 50 wt. % on a free phenol basis;

(b) the taste-masking polymer is employed in an amount in a range of from about 1 to about 25 wt. %;

(c) the superdisintegrant is employed in an amount in a range of from about 1 to about 15 wt. %; and

(d) the compression aid is employed in an amount in a range of from about 5 to about 50 wt. %;

(e) the water soluble filler is employed in an amount in a range of from about 5 to about 75 wt. %; and

(f) the lubricant is employed in an amount in a range of from about 0.1 to about 10 wt. %.

Another embodiment of the present invention (Embodiment E10) is a pharmaceutical oral dosage form described in Embodiment E9, which further comprises a sweetening agent, a taste modifier, and optionally a flavoring agent, wherein:

(g) the sweetening agent is employed in an amount in a range of from about 0.1 to about 10 wt. %;

(h) the taste modifier is employed in an amount in a range of from about 0.1 to about 2 wt. %; and

(i) the flavoring agent is employed in an amount in a range of from 0 to about 10 wt. %.

Another embodiment of the present invention (Embodiment E11) is the pharmaceutical oral dosage form as originally defined above, which further comprises: (d) a compression aid; (e) a water soluble filler; and (f) a lubricant; wherein:

(b) the taste-masking polymer comprises a cellulose polymer, a vinyl carboxylate-alkylene glycol copolymer, an acrylic polymer, a methacrylic polymer, or an acrylic-methacrylic copolymer;

(c) the superdisintegrant comprises croscarmellose sodium, crospovidone, or sodium starch glycolate;

(d) the compression aid comprises lactose, sucrose, anhydrous dibasic calcium phosphate, dibasic calcium phosphate dihydrate, tribasic calcium phosphate, calcium sulfate, carboxymethylcellulose calcium, microcrystalline cellulose, or powdered cellulose;

(e) the water soluble filler comprises mannitol, glucose, dextrose, or sucrose; and

(e) the lubricant comprises a metal stearate, a metal stearyl fumarate, or stearic acid.

Additional embodiments of the present invention include the pharmaceutical oral dosage form as just described in Embodiment E11 incorporating one or more of the features (i) to (v) as follows:

(i-a) the taste-masking polymer comprises hydroxypropylmethylcellulose (BPMC), hydroxypropylcellulose (HPC), ethylcellulose, cellulose acetate, a vinyl acetate-ethylene glycol copolymer, or a methacrylic copolymer;

(i-b) the taste-masking polymer comprises an aminoalkyl methacrylate copolymer;

(i-c) the taste-masking polymer comprises Eudragit® E100 or Eudragit® E PO; or

(i-d) the taste-masking polymer comprises Eudragit® E PO;

(ii-a) the superdisintegrant comprises crospovidone;

(iii-a) the compression aid comprises microcrystalline cellulose; or

(iii-b) the compression aid comprises Avicel PH-102;

(iv-a) the water soluble filler comprises mannitol; and

(v-a) the lubricant comprises magnesium stearate, sodium stearyl fumarate, or a combination thereof; or

(v-b) the lubricant comprises magnesium stearate;

Still another embodiment of the present invention (Embodiment E12) is the pharmaceutical oral dosage form as described in Embodiment E11, which further comprises: (g) a sweetening agent, (h) a taste modifier, and optionally (i) a flavoring agent; wherein

(g) the sweetening agent comprises aspartame, acesulfame potassium, sodium saccharin, or sucralose; and

(h) the taste modifier comprises monoammonium glycyrrhizinate, sorbitol, maltose, maltodextrin, dextrose, or fructose.

Additional embodiments of the present invention include the pharmaceutical oral dosage form as just described in Embodiment E12 incorporating one or more of the features (i) to (v) as just set forth above and features (vi) and (vii) as follows:

(vi) the sweetening agent is aspartame; and

(vii) the taste modifier is monoammonium glycyrrhizinate.

Another embodiment of the present invention (Embodiment E13) is the oral dosage form as described in Embodiment E12, wherein the oral dosage form is granules prepared by a process which comprises dry blending the ingredients and granulating the dry blend (e.g., dry granulating by rolling the dry blend to form a compact, and then milling the compact to form granules).

Another embodiment of the present invention (Embodiment E14) is the oral dosage form as described in Embodiment E12, wherein the oral dosage form is a tablet (e.g., an ODT) prepared by a process which comprises dry blending the compound or its pharmaceutically acceptable salt, the taste-masking polymer, the compression aid, a first portion of the lubricant, the taste modifier, a first portion of the sweetening agent, and a first portion of the flavoring agent (if employed); granulating the dry blend (e.g., dry granulating by rolling the dry blend to form a compact, and then milling the compact to form granules); mixing the resulting granules with the superdisintegrant, the water soluble filler, the remaining portion of the lubricant, the remaining portion of the sweetening agent, and the remaining portion of the flavoring agent (if employed); and compressing the mixture. In the dry blending step of this embodiment, all of the ingredients except the lubricant can first be added to the blender until well mixed, and then the lubricant can be added and the admixture blended until the lubricant is well mixed therewith. In the step of mixing the granules, some of the remaining portion of the lubricant can first be mixed with the granules and then the other ingredients (i.e., the water soluble filler, the superdisintegrant, the rest of the remaining portion of the lubricant, the remaining portion of the sweetening agent, and the remaining portion of the flavoring agent—if employed) can be mixed with the lubricated granules.

In an aspect of Embodiment E14, the tablet resulting from compression of the mixture is an orally disintegrating tablet (ODT) with a hardness in a range of from about 2 to about 4 kiloponds.

In Embodiment E14, the ingredients which are dry blended can be added to the blender concurrently or at different times in any order. Furthermore, each particular ingredient can be added all at once or added in two or more portions at the same or different times in the dry blending step.

Still another embodiment of the present invention (Embodiment E15) is the oral dosage form as described in Embodiment E12, wherein the oral dosage form is a tablet (e.g., a chewable tablet) prepared by a process which comprises dry blending the compound or its pharmaceutically acceptable salt, the taste-masking polymer, the compression aid, the lubricant, the sweetening agent, the taste modifier, the flavoring agent (if employed), the superdisintegrant, and the water soluble filler; granulating the dry blend (e.g., dry granulating by rolling the dry blend to form a compact, and then milling the compact to form granules); and compressing the granules. In an aspect of this embodiment, the tablet resulting from compression of the granules is a chewable tablet having a hardness in a range of from about 6 to about 9 kiloponds.

Another embodiment of the present invention (Embodiment E16) is the pharmaceutical oral dosage form as originally defined above or as defined in any preceding embodiment, wherein the carboxamide compound is in the form of a base salt (e.g., an alkali metal salt such as a Na salt or a K salt).

The base salt of the carboxamide compound employed in the pharmaceutical oral dosage form of the present invention is a pharmaceutically acceptable salt. The term “pharmaceutically acceptable salt” refers herein to a salt which possesses the effectiveness of the parent compound and which is not biologically or otherwise undesirable (e.g., is neither toxic nor otherwise deleterious to the recipient thereof). Suitable base salts include salts formed by reaction of the carboxamide compound with a base, including, for example, alkali metal salts (e.g., sodium or potassium salts), alkaline earth metal salts (e.g., calcium or magnesium salts) and ammonium salts. Alkali metal salts of the compounds can be formed by treating the compound dissolved in a suitable solvent with an aqueous solution of the alkali metal hydroxide (e.g., NaOH or KOH).

Another embodiment of the present invention (Embodiment E17) is the pharmaceutical oral dosage form as originally defined or as defined in any preceding embodiment, wherein the carboxamide compound is a compound of Formula I:

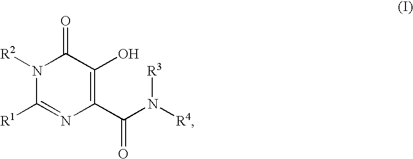

wherein R¹ is C₁₋₆ alkyl substituted with:

(1) N(R^{A})—C(═O)—N(R^{C})R^{D},

(2) N(R^{A})—C(═O)—C₁₋₆ alkylene-N(R^{C})R^{D},

(3) N(R^{A})SO₂R^{B},

(4) N(R^{A})SO₂N(R^{C})R^{D},

(5) N(R^{A})—C(═O)—C₁₋₆ alkylene-SO₂R^{B},

(6) N(R^{A})—C(—O)—C₁₋₆ alkylene-SO₂N(R^{C})R^{D},

(7) N(R^{A})C(═O)C(—O)N(R^{C})R^{D},

(8) N(R^{A})—C(—O)—Het,

(9) N(R^{A})C(═O)C(═O)—HetA, or

(10) HetB;

R² is C₁₋₆ alkyl;
or alternatively R¹ and R² are linked together such that the compound of Formula I is a compound of Formula II:

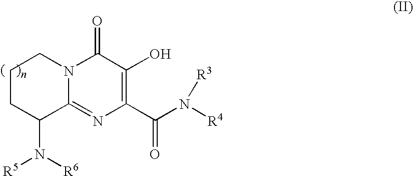

R³ is H or C₁₋₆ alkyl;
R⁴ is C₁₋₆ alkyl substituted with an aryl, which is optionally substituted with from 1 to 4 substituents each of which is independently halogen, —OH, —C₁₋₄ alkyl, —C₁₋₄ alkyl-ORA, —C₁₋₄ haloalkyl, —O—C₁₋₄ alkyl, —O—C₁₋₄ haloalkyl, —CN, —NO₂, —N(R^{A})R^{B}, —C₁₋₄ alkyl-N(R^{A})R^{B}, —C(═O)N(R^{A})R^{B}, —C(═O)R^{A}, —CO₂R^{A}, —C₁₋₄ alkyl-CO₂R^{A}, —OCO₂R^{A}, —SR^{A}, —S(═O)R^{A}, —SO₂R^{A}, —N(R^{A})SO₂R^{B}, —SO₂N(R^{A})R^{B}, —N(R^{A})C(═O)R^{B}, —N(R^{A})CO₂R^{B}, —C₁₋₄ alkyl-N(R^{A})CO₂R^{B}, methylenedioxy attached to two adjacent ring carbon atoms, phenyl, or —C₁₋₄ alkyl-phenyl;

### R⁵ is:

(1) N(R^{A})—C(═O)—N(R^{C})R^{D},

(2) N(R^{A})—C(═O)—C₁₋₆ alkylene-N(R^{C})R^{D},

(3) N(R^{A})SO₂R^{B},

(4) N(R^{A})SO₂N(R^{C})R^{D},

(5) N(R^{A})—C(O)—C₁₋₆ alkylene-SO₂R^{B},

(6) N(R^{A})—C(═O)—C₁₋₆ alkylene-SO₂N(R^{C})R^{D},

(7) N(R^{A})C(═O)C(—O)N(R^{C})R^{D},

(8) N(R^{A})—C(—O)—HetA, or

(9) N(R^{A})C(═O)C(═O)—HetA;

R⁶ is —H or —C₁₋₆ alkyl;
n is an integer equal to 1 or 2;
each R^{A} is independently —H or —C₁₋₆ alkyl;
each R^{B} is independently —H or —C₁₋₆ alkyl;
R^{C} and R^{D} are each independently —H or —C₁₋₆ alkyl, or together with the nitrogen to which they are attached form a saturated 5- or 6-membered heterocyclic ring optionally containing a heteroatom in addition to the nitrogen attached to R^{C} and R^{D} selected from N, O, and S, where the S is optionally oxidized to S(O) or S(O)₂, and wherein the saturated heterocyclic ring is optionally substituted with 1 or 2 C₁₋₆ alkyl groups;
HetA is a 5- or 6-membered heteroaromatic ring containing from 1 to 4 heteroatoms independently selected from N, O and S, wherein the heteroaromatic ring is optionally substituted with 1 or 2 substituents each of which is independently —C₁₋₄ alkyl, —C₁₋₄ haloalkyl, —O—C₁₋₄ alkyl, —O—C₁₋₄ haloalkyl, or —CO₂R^{A}; and
HetB is a 5- to 7-membered saturated heterocyclic ring containing from 1 to 4 heteroatoms independently selected from N, O and S, wherein each S is optionally oxidized to S(O) or S(O)₂, and the heterocyclic ring is optionally substituted with from 1 to 3 substituents each of which is independently halogen, —C₁₋₄ alkyl, —C₁₋₄ fluoroalkyl, —C(O)—C₁₋₄ alkyl, or —C₁₋₄ alkyl substituted with OH.

As used herein, the term “alkyl” refers to any linear or branched chain alkyl group having a number of carbon atoms in the specified range. Thus, for example, “C₁₋₆ alkyl” (or “C₁-C₆ alkyl”) refers to any of the hexyl alkyl and pentyl alkyl isomers as well as n-, iso-, sec- and t-butyl, n- and isopropyl, ethyl and methyl. As another example, “C₁₋₄ alkyl” refers to n-, iso-, sec- and t-butyl, n- and isopropyl, ethyl and methyl.

The term “alkylene” refers to any linear or branched chain alkylene group (or alternatively “alkanediyl”) having a number of carbon atoms in the specified range. Thus, for example, “—C₁₋₆ alkylene-” refers to any of the C₁ to C₆ linear or branched alkylenes. A class of alkylenes of particular interest with respect to the invention is —(CH₂)₁₋₆—, and sub-classes of particular interest include —(CH₂)₁₋₄—, —(CH₂)₁₋₃—, —(CH₂)₁₋₂—, and —CH₂—. Also of interest is the alkylene —CH(CH₃)—.

The term “halogen” (or “halo”) refers to fluorine, chlorine, bromine and iodine (alternatively referred to as fluoro, chloro, bromo, and iodo).

The term “haloalkyl” refers to an alkyl group as defined above in which one or more of the hydrogen atoms has been replaced with a halogen (i.e., F, Cl, Br and/or I). Thus, for example, “C₁₋₆ haloalkyl” (or “C₁-C₆ haloalkyl”) refers to a C₁ to C₆ linear or branched alkyl group as defined above with one or more halogen substituents. The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.).

The term “aryl” refers to (i) phenyl or (ii) a 9- or 10-membered bicyclic, fused carbocylic ring system in which at least one ring is aromatic. Aryl is typically phenyl or naphthyl, and is more typically phenyl.

The term “HetA” refers to an optionally substituted a 5- or 6-membered heteroaromatic ring containing from 1 to 4 heteroatoms independently selected from N, O and S. In one embodiment, HetA is an optionally substituted heteroaromatic ring selected from the group consisting of pyridinyl, pyrrolyl, pyrazinyl, pyrimidinyl, pyridazinyl, triazinyl, furanyl, thienyl, imidazolyl, pyrazolyl, triazolyl, tetrazolyl, oxazolyl, isooxazolyl, thiazoly, isothiazolyl, and oxadiazolyl; wherein the optional substitution is with 1 or 2 substituents each of which is independently —C₁₋₄ alkyl, —C₁₋₄ haloalkyl, —O—C₁₋₄ alkyl, —O—C₁₋₄ haloalkyl, or —CO₂—C₁₋₄alkyl. It is understood that HetA can be attached to the rest of the compound of Formula I at any ring atom (i.e., any carbon atom or any heteroatom) provided that a stable compound results.

The term “HetB” refers to an optionally substituted a 5- to 7-membered saturated heterocyclic ring containing from 1 to 4 heteroatoms independently selected from N, O and S. In one embodiment, HetB is an optionally substituted saturated heterocyclic ring selected from the group consisting of pyrrolidinyl, imidazolidinyl, piperidinyl, piperazinyl, morpholinyl, thiomorpholinyl, thiazinanyl, and tetrahydropyranyl, wherein the optional substitution is with 1 or 2 substituents each of which is independently —C₁₋₄ alkyl, —C₁₋₄ haloalkyl, —C(O)CF₃, —C(O)CH₃, or —CH₂CH₂OH. It is understood that HetA can be attached to the rest of the compound of Formula I at any ring atom (i.e., any carbon atom or any heteroatom) provided that a stable compound results. In another embodiment, HetB is selected from the group consisting of

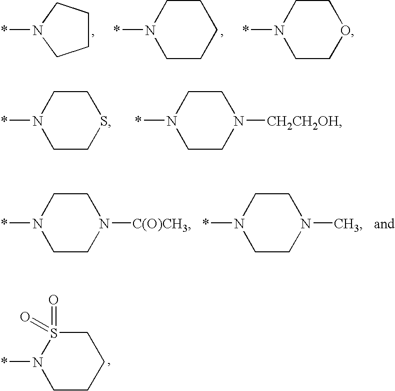

wherein * denotes the point of attachment to the rest of the molecule.

In the compound of Formula I, R^{C} and R^{D} together with the nitrogen to which they are attached can form a saturated 5- or 6-membered heterocyclic ring optionally containing a heteroatom in addition to the nitrogen attached to R^{C} and R^{D} selected from N, O, and S, where the S is optionally oxidized to S(O) or S(O)₂, and wherein the saturated heterocyclic ring is optionally substituted with 1 or 2 C₁₋₆ alkyl groups. In one embodiment, the saturated heterocyclic ring formed by R^{C} and R^{D} and the nitrogen to which they are attached is selected from the group consisting of 4-morpholinyl, 4-thiomorpholinyl, 1-piperidinyl, 1-piperazinyl optionally substituted with C₁₋₄ alkyl (e.g., methyl), and 1-pyrrolidinyl.

As noted earlier, unless expressly stated to the contrary, all ranges cited herein are inclusive. Thus, for example, a heterocyclic ring described as containing from “1 to 4 heteroatoms” means the ring can contain 1, 2, 3 or 4 heteroatoms.

When any variable (e.g., R^{A} and R^{B}) occurs more than one time in Formula I or in any other formula depicting and describing a compound whose salt can be employed in pharmaceutical formulations of the invention, its definition on each occurrence is independent of its definition at every other occurrence. Also, combinations of substituents and/or variables are permissible to the extent such combinations result in stable compounds.

A “stable” compound is a compound which can be prepared and isolated and whose structure and properties remain or can be caused to remain essentially unchanged for a period of time sufficient to allow use of the compound for the purposes described herein (e.g., use in a pharmaceutical oral dosage form of the invention).

As a result of the selection of substituents and substituent patterns, certain of the compounds of Formula I can have asymmetric centers and can occur as mixtures of stereoisomers, or as individual diastereomers, or enantiomers. All isomeric forms of these compounds, whether individually or in mixtures, can be employed in pharmaceutical oral dosage forms of the present invention.

Compounds of Formula I can also exist as tautomers due to keto-enol tautomerism. All tautomers of the hydroxypyrimidinone compounds of Formula I, both singly and in mixtures, can be employed in pharmaceutical oral dosage forms of the present invention.

Additional embodiments of the present invention include the following:

(E17a) a pharmaceutical oral dosage form as set forth in Embodiment E17, wherein the carboxamide compound of Formula I is in the form of a pharmaceutically acceptable base salt;

(E17b) a pharmaceutical oral dosage form as set forth in Embodiment E17, wherein the carboxamide compound of Formula I is in the form of a pharmaceutically acceptable alkali metal salt;

(E17c) a pharmaceutical oral dosage form as set forth in Embodiment E17, wherein Compound I is Compound A, which is:

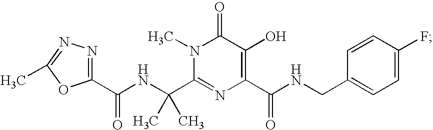

(E17d) a pharmaceutical oral dosage form as set forth in Embodiment E17, wherein Compound I is Compound A in the form of a pharmaceutically acceptable base salt;

(E17e) a pharmaceutical oral dosage form as set forth in Embodiment E17, wherein Compound I is Compound A in the form of a pharmaceutically acceptable alkali metal salt of Compound A;

(E17f) a pharmaceutical oral dosage form as set forth in Embodiment E17, wherein Compound I is Compound A in the form of a potassium salt;

(E17g) a pharmaceutical oral dosage form as set forth in Embodiment E17, wherein Compound I is Compound A in the form of a crystalline potassium salt; and

(E17h) a pharmaceutical oral dosage form as set forth in Embodiment E17, wherein Compound I is Compound A in the form of a crystalline potassium salt, wherein the K salt is an anhydrous crystalline salt characterized by an X-ray powder diffraction pattern obtained using copper K_{α} radiation (i.e., the radiation source is a combination of Cu K_{α1} and K_{α2} radiation) which comprises 20Θ values (i.e., reflections at 2Θ values) in degrees of 5.9, 12.5, 20.0, 20.6 and 25.6 (designated herein as the Form 1 K salt of Compound A).

Crystalline potassium salts of Compound A can be prepared by dissolving Compound A in an alcohol (e.g., ethanol) or an alcohol-water mixture (e.g., ethanol-water), adding an aqueous solution of KOH to the solution of Compound A and optionally adding additional alcohol, and then allowing the crystalline potassium salt of Compound A to form from the admixture. The admixture can optionally be seeded with crystalline Compound A K salt to promote/assist crystal formation. The preparation of crystalline potassium salts of Compound A is further described in a patent application entitled “Potassium Salt of an HIV Integrase Inhibitor”, International Application No. PCT/IUS05/43781, filed Dec. 2, 2005, published as WO ______, incorporated herein by reference in its entirety.

The present invention also includes a pharmaceutical oral dosage form (alternatively referred to herein as Dosage Form Q) which is a tablet or granules, which comprises:

(a) Compound A in the form of an alkali metal salt, wherein Compound A is

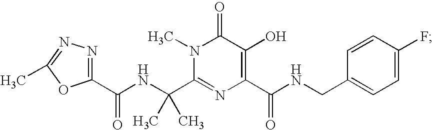

(b) a taste-masking polymer which is an aminoalkyl methacrylate copolymer; and

(c) a superdisintegrant which is crospovidone, croscarmellose sodium, or sodium starch glycolate.

An embodiment of the present invention (Embodiment Q1) is Dosage Form Q as just set forth above, which further comprises:

(d) a compression aid which is lactose, sucrose, anhydrous dibasic calcium phosphate, dibasic calcium phosphate dihydrate, tribasic calcium phosphate, calcium sulfate, carboxymethylcellulose calcium, microcrystalline cellulose, or powdered cellulose;

(e) a water soluble filler which is mannitol;

(f) a lubricant which is magnesium stearate, sodium stearyl fumarate, or stearic acid;

(g) optionally a sweetening agent which is aspartame, acesulfame potassium, sodium saccharin, or sucralose;

(h) optionally a taste modifier which is monoammonium glycyrrhizinate, sorbitol, maltose, maltodextrin, dextrose, or fructose; and

(i) optionally a flavoring agent.

In an aspect of this embodiment, the dosage form includes the sweetening agent and the taste masking agent and optionally includes the flavoring agent. In another aspect of this embodiment, the dosage form includes the sweetening agent, the taste masking agent, and the flavoring agent.

Another embodiment of the present invention (Embodiment Q1a) is the pharmaceutical oral dosage form described in Embodiment Q1, wherein the dosage form is a tablet prepared by a process comprising:

(A) dry blending the alkali metal salt of Compound A, the aminoalkyl methacrylate copolymer, the superdisintegrant, the compression aid, the water soluble filler, the lubricant, optionally the sweetening agent, optionally the taste modifier, and optionally the flavoring agent;

(B) granulating the dry blend (e.g., rolling the dry blend to form a compact, and then milling the compact to form granules); and

(C) compressing the granules to obtain the tablet.

In an aspect of this embodiment, the tablet is a chewable tablet.

Another embodiment of the present invention (Embodiment Q1b) is the tablet described in Embodiment Q1a, wherein

(a) the alkali metal salt of Compound A is employed in an amount in a range of from about 1 to about 50 wt. % on a free phenol basis;

(b) the aminoalkyl methacrylate copolymer is employed in an amount in a range of from about 1 to about 25 wt. %;

(c) the superdisintegrant is employed in an amount in a range of from about 1 to about 15 wt. %;

(d) the compression aid is employed in an amount in a range of from about 5 to about 50 wt. %;

(e) the mannitol is employed in an amount in a range of from about 5 to about 75 wt. %;

(f) the lubricant is employed in an amount in a range of from about 0.1 to about 10 wt. %;

(g) the sweetening agent is employed in an amount in a range of from 0 to about 10 wt. %;

(h) the taste modifier is employed in an amount in a range of from 0 to about 2 wt. %; and

(i) the flavoring agent is employed in an amount in a range of from 0 to about 10 wt. %.

Another embodiment of the present invention (Embodiment Q1c) is the tablet described in Embodiment Q1b, wherein

(a) the alkali metal salt of Compound A is a potassium salt of Compound A (e.g., a crystalline potassium salt of Compound A such as a Form 1 crystalline potassium salt of Compound A);

(b) the aminoalkyl methacrylate copolymer is Eudragit® E100 or Eudragit® E PO;

(c) the superdisintegrant is crospovidone;

(d) the compression aid is microcrystalline cellulose; and

(e) the lubricant is magnesium stearate.

Another embodiment of the present invention (Embodiment Q1d) is the tablet described in Embodiment Q1c, wherein

(a) the potassium salt of Compound A is employed in an amount in a range of from about 5 to about 25 wt. % on a free phenol basis;

(b) the Eudragit is employed in an amount in a range of from about 2 to about 15 wt. %;

(c) the crospovidone is employed in an amount in a range of from about 1 to about 5 wt. %;

(d) the microcrystalline cellulose is employed in an amount in a range of from about 5 to about 25 wt. %;

(e) the mannitol is employed in an amount in a range of from about 30 to about 60 wt. %;

(f) the magnesium stearate is employed in an amount in a range of from about 0.5 to about 3 wt. %;

(g) the sweetening agent is employed in an amount in a range of from 0 to about 3 wt. %;

(h) the taste modifier is employed in an amount in a range of from 0 to about 2 wt. %; and

(i) the flavoring agent is employed in an amount in a range of from 0 to about 3 wt. %.

In an aspect of this embodiment, (g) the sweetening agent is employed in an amount in a range of from 0.1 to about 3 wt. %; (h) the taste modifier is employed in an amount in a range of from 0.1 to about 2 wt. %; and (i) the flavoring agent is employed in an amount in a range of from 0.1 to about 3 wt. %.

Another embodiment of the present invention (Embodiment Q1e) is the tablet described in any of the four immediately preceding embodiments Q1a, Q1b, Q1c, and Q1d, wherein the tablet formed by compressing the mixture has a hardness in a range of from about 6 to about 9 kiloponds.

Another embodiment of the present invention (Embodiment Q1f) is the tablet described in any of the five immediately preceding embodiments Q1a, Q1b, Q1c, Q1d and Q1e, wherein the amount of the potassium salt of Compound A is in a range of from about 20 to about 100 mg per tablet on a free phenol basis.

Another embodiment of the present invention (Embodiment Q2a) is the pharmaceutical oral dosage form described in Embodiment Q1, wherein the dosage form is a tablet prepared by a process comprising:

(A) dry blending the alkali metal salt of Compound A, the aminoalkyl methacrylate copolymer, the compression aid, a first portion of the lubricant, all or a first portion of the sweetening agent (if employed), all or a first portion of the taste modifier (if employed), and all or a first portion of the flavoring agent (if employed);

(B) granulating the dry blend (e.g., rolling the dry blend to form a compact, and then milling the compact to form granules);

(C) mixing the granules with the superdisintegrant, the mannitol, the remaining portion of the lubricant, any remaining portion of the sweetening agent (if employed), any remaining portion of the taste modifier (if employed), and any remaining portion of the flavoring agent (if employed); and

(D) compressing the mixture to obtain the tablet.

In an aspect of this embodiment, the tablet is an ODT.

Another embodiment of the present invention (Embodiment Q2b) is the tablet described in Embodiment Q2a, wherein

(a) the alkali metal salt of Compound A is employed in an amount in a range of from about 1 to about 50 wt. % on a free phenol basis;

(b) the aminoalkyl methacrylate copolymer is employed in an amount in a range of from about 1 to about 25 wt. %;

(c) the superdisintegrant is employed in an amount in a range of from about 1 to about 15 wt. %;

(d) the compression aid is employed in an amount in a range of from about 5 to about 50 wt.%;

(e) the mannitol is employed in an amount in a range of from about 5 to about 75 wt. %;

(f) the lubricant is employed in an amount in a range of from about 0.1 to about 5 wt. %;

(g) the sweetening agent is employed in an amount in a range of from 0 to about 5 wt. %;

(h) the taste modifier is employed in an amount in a range of from 0 to about 2 wt. %; and

(i) the flavoring agent is employed in an amount in a range of from 0 to about 5 wt. %.

Another embodiment of the present invention (Embodiment Q2c) is the tablet described in Embodiment Q2b, wherein

(a) the alkali metal salt of Compound A is a potassium salt of Compound A (e.g., a crystalline potassium salt of Compound A such as a Form 1 crystalline potassium salt of Compound A);

(b) the aminoalkyl methacrylate copolymer is Eudragit® E100 or Eudragit® E PO;

(c) the superdisintegrant is crospovidone;

(d) the compression aid is microcrystalline cellulose; and

(f) the lubricant is magnesium stearate.

Another embodiment of the present invention (Embodiment Q2d) is the tablet described in Embodiment Q2c, wherein

(a) the potassium salt of Compound A is employed in an amount in a range of from about 5 to about 25 wt. % on a free phenol basis;

(b) the Eudragit is employed in an amount in a range of from about 2 to about 15 wt. %;

(c) the crospovidone is employed in an amount in a range of from about 1 to about 5 wt. %;

(d) the microcrystalline cellulose is employed in an amount in a range of from about 5 to about 25 wt. %;

(e) the mannitol is employed in an amount in a range of from about 30 to about 60 wt. %;

(f) the magnesium stearate is employed in an amount in a range of from about 0.5 to about 2 wt. %;

(g) the sweetening agent is employed in an amount in a range of from 0 to about 2 wt. %;

(h) the taste modifier is each employed in an amount in a range of from 0 to about 1 wt. %; and

(i) the flavoring agent is employed in an amount in a range of from 0 to about 2 wt. %.

In an aspect of this embodiment, (g) the sweetening agent is employed in an amount in a range of from 0.1 to about 2 wt. %; (h) the taste modifier is employed in an amount in a range of from about 0.1 to about 1 wt. %; and (i) the flavoring agent is employed in an amount in a range of from 0.1 to about 2 wt. %.

Another embodiment of the present invention (Embodiment Q2e) is the tablet described in any of the four immediately preceding embodiments Q2a, Q2b, Q2c, and Q2d, wherein the tablet formed by compressing the mixture has a hardness in a range of from about 2 to about 4 kiloponds.

Another embodiment of the present invention (Embodiment Q2f) is the tablet described in any of the five immediately preceding embodiments Q2a, Q2b, Q2c, Q2d and Q2e, wherein the amount of the potassium salt of Compound A is in a range of from about 20 to about 100 mg per tablet on a free phenol basis.

The present invention also includes methods for preparing the pharmaceutical oral dosage form as originally defined above or as defined in any of the foregoing embodiments. In particular, the present invention includes the preparative methods per se described above in various embodiments of the oral dosage form of the invention such as the processes per se included in Embodiments E1, E2, E3, E5, E13, E14, E15, Q1a and Q2a. More generally, granules of the present invention can be obtained via granulation, wherein the overall particle size of a formulation is increased through the permanent aggregation of smaller particles. Wet or dry granulation can be employed. Wet granulation can be accomplished, for example, by wetting a well-mixed blend of the dry ingredients with sufficient solvent (e.g., water or water with an alcohol co-solvent) to moisten the dry blend such that particles in the blend tack to one another to form larger particles, and then sieving, comminuting, or otherwise manipulating the size of the particles. Once formed, the resulting wet granulate can then be dried and milled into suitably sized particles (i.e., granules). The granules can then be used per se, or they can be directly compressed into tablets, or they can be blended with a lubricant and optionally one or more other ingredients and then compressed into tablets.

Dry granulation is preferred to wet granulation, since at least certain of the hydroxypyrimidinone carboxamides employed in the pharmaceutical oral dosage forms of the invention (e.g., Compound A) are susceptible to hydrolytic degradation. Dry granulation can be accomplished, for example, by dry blending the ingredients and then compressing the blended mixture into slugs or rolling the blended mixture into a compact. The slugs or compact can then be sized (e.g., by passing through a mesh screen or a comminuting mill) to afford the dry granules, which can then used as is, directly compressed into tablets, or blended with lubricant and optionally one or more other ingredients and compressed into tablets.

Technology and equipment suitable for preparing the oral dosage forms of the present invention (e.g., compressed tablets and unit doses of granules) are described, for example, in Remington's Pharmaceutical Sciences, 18^{th} edition, edited by A. R. Gennaro, 1990, Chapter 89.

The pharmaceutical oral dosage forms of the present invention are useful in the inhibition of HIV integrase, the treatment or prophylaxis of infection by HIV, and the treatment, prophylaxis, or the delay in the onset of consequent pathological conditions such as AIDS. Treating AIDS, the prophylaxis of AIDS, delaying the onset of AIDS, treating HIV infection, or prophylaxis of HIV infection is defined as including, but not limited to, treatment or prophylaxis of a wide range of states of HIV infection: AIDS, ARC, both symptomatic and asymptomatic, and actual or potential exposure to HIV. For example, the compositions of this invention are useful in treating or prophylaxis of infection by HIV after suspected past exposure to HIV by such means as blood transfusion, exchange of body fluids, bites, accidental needle stick, or exposure to patient blood during surgery.

The present invention includes a method for inhibiting HIV integrase in a subject in need thereof which comprises orally administering to the subject a pharmaceutical dosage form of the present invention as originally defined above. The invention also includes a method for the treatment or prophylaxis of HIV infection or for the treatment, prophylaxis, or delay the onset of AIDS in a subject in need thereof, which comprises orally administering to the subject a pharmaceutical dosage form of the invention as originally defined above. In these methods, the oral dosage form of the present invention can optionally be employed in combination with one or more anti-HIV agents selected from HIV antiviral agents, anti-infective agents, and immunomodulators, where it is understood the other anti-HIV agents can be given orally or by another mode of administration. Embodiments of these methods include the methods as just described wherein the pharmaceutical oral dosage form of the invention is a formulation as set forth in any one of the foregoing embodiments thereof (including, inter alia, the Dosage Form Q).

The term “subject” (used interchangeably herein with “patient”) refers to an animal, preferably a mammal, most preferably a human, who has been the object of treatment, observation or experiment.

When a pharmaceutical oral dosage form of the present invention is employed or administered in combination with another agent (e.g., when the Dosage Form Q is administered in combination with an anti-HIV agent), the dosage form and agent can be administered separately or together, and when administered separately, the dosage form and agent can be given concurrently or at different times (e.g., alternately).

The present invention also includes a pharmaceutical oral dosage form as originally defined and described in the Summary of the Invention (i) for use in, (ii) for use as a medicament for, or (iii) for use in the preparation of a medicament for: (a) the inhibition of HIV integrase, (b) treatment or prophylaxis of infection by HIV, or (c) treatment, prophylaxis of, or delay in the onset of AIDS. Embodiments of these uses include the uses as just described wherein the oral dosage form of the invention as originally defined is replaced with the above-described embodiments thereof (which include, inter alia, Dosage Form Q). In these uses, the oral dosage forms of the present invention can optionally be employed in combination with one or more anti-HIV agents selected from HIV antiviral agents, anti-infective agents, and immunomodulators.

An “anti-HIV agent” is any agent which is directly or indirectly effective in the inhibition of HIV integrase or another enzyme required for HIV replication or infection, the treatment or prophylaxis of HIV infection, and/or the treatment, prophylaxis or delay in the onset of AIDS. It is understood that an anti-HIV agent is effective in treating, preventing, or delaying the onset of HIV infection or AIDS and/or diseases or conditions arising therefrom or associated therewith. For example, the compounds of this invention may be effectively administered, whether at periods of pre-exposure and/or post-exposure, in combination with effective amounts of one or more anti-HIV agents selected from HIV antiviral agents, immunomodulators, antiinfectives, and vaccines useful for treating HIV infection or AIDS, such as those disclosed in Table 1 of WO 01/38332 or in the Table in WO 02/30930, both of these tables herein incorporated by reference. Suitable HIV antivirals for use in combination with the compounds of the present invention include, for example, those listed in Table A as follows:

TABLE A

| Name | Type |
|---|---|
| abacavir, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Reyataz ® | PI |
| AZT, zidovudine, Retrovir ® | nRTI |
| capravirine | nnRTI |
| ddC, zalcitabine, dideoxycytidine, Hivid ® | nRTI |
| ddI, didanosine, dideoxyinosine, Videx ® | nRTI |
| delavirdine, Rescriptor ® | nnRTI |
| efavirenz, Sustiva ®, Stocrin ® | nnRTI |
| emtricitabine, FTC, Emtriva ® | nRTI |
| emtricitabine + tenofovir disoproxil, Truvada ® | nRTI |
| emvirine, Coactinon ® | nnRTI |
| enteric coated didanosine, Videx EC ® | nRTI |
| enfuvirtide, Fuzeon ® | FI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| lopinavir | PI |
| lopinavir + ritonavir, Kaletra ® | PI |
| nelfinavir, Viracept ® | PI |
| nevirapine, Viramune ® | nnRTI |
| ritonavir, Norvir ® | PI |
| saquinavir, Invirase ®, Fortovase ® | PI |
| stavudine, d4T, didehydrodeoxythymidine, Zerit ® | nRTI |
| tenofovir disoproxil fumarate, Viread ® | nRTI |
| tipranavir, Aptivus ® | PI |

FI = fusion inhibitor; PI = protease inhibitor; nRTI = nucleoside reverse transcriptase inhibitor; nnRTI = non-nucleoside reverse transcriptase inhibitor. Some of the drugs listed in the table are used in a salt form; e.g., indinavir sulfate, atazanvir sulfate, nelfinvavir mesylate.

The pharmaceutical oral dosage forms of the present invention can be administered so as to provide the active ingredient in a dosage range of from about 0.001 to about 1000 mg/kg of mammal (e.g., human) body weight per day in a single dose or in divided doses. One preferred dosage range is from about 0.01 to about 500 mg/kg body weight per day in a single dose or in divided doses. Another preferred dosage range is from about 0.1 to about 100 mg/kg body weight per day in single or divided doses.

The oral dosage forms of the invention are suitably in the form of granules or a tablet, wherein the tablet or dose of granules contains from about 1 to about 1000 milligrams of the active ingredient, particularly 1, 5, 10, 15, 20, 25, 50, 75, 100, 150, 200, 250, 300, 400, 500, 600, 700, 800, 900 and 1000 milligrams of the active ingredient for the symptomatic adjustment of the dosage to the patient to be treated. In particular, pharmaceutical oral dosage forms of the present invention containing a potassium salt of Compound A (e.g., a crystalline K salt such as the Form 1 crystalline K salt) are preferably dosed to adult humans in an amount of from 100 mg to 600 mg of Compound A twice per day; e.g., 200 mg, 400 mg, or 600 mg twice per day. Oral dosage forms containing a potassium salt of Compound A (e.g., a crystalline K salt such as the Form 1 crystalline K salt) can be dosed to children in an amount suitable for their age and body weight and size. It is believed that doses suitable for children are in a range of from about 25 mg to 200 mg of Compound A twice per day; e.g., 25 mg, 50 mg, or 200 mg twice per day.

The specific dose level and frequency of dosage for any particular patient will depend upon a variety of factors including the activity of the specific drug compound employed, the metabolic stability and length of action of that compound, the age, body weight, general health, sex, diet, mode and time of administration, rate of excretion, drug combination, the severity of the particular condition, and the host undergoing therapy. The appropriate dose level of a particular drug suitable for a particular patient can be determined by the person of ordinary skill in the art without undue experimentation.

Abbreviations used herein include the following: ACN=acetonitrile; AIDS=acquired immunodeficiency syndrome; ARC=AIDS related complex; Cbz benzyloxycarbonyl; DIEA=diisopropylethylamine; DMADC=dimethylacetylene dicarboxylate; DMFc N,N-dimethylformamide; DMSO=dimethylsulfoxide; DSC=differential scanning calorimetry; EtOH=ethanol; Eq.=equivalent(s); RN=human immunodeficiency virus; HPLC=high-performance liquid chromatography; HPMC=hydroxypropylmethylcellulose; IPA=isopropyl alcohol; KF=Karl Fisher titration for water; MeOH=methanol; MSA=methanesulfonic acid; MTBE=methyl tertiary butyl ether; MW=molecular weight; NMM=N-methylmorpholine; NMR=nuclear magnetic resonance; ODT=orally disintegrating tablet; TG=thermogravimetric; THF=tetrahydrofuran; XRPD=x-ray powder diffraction.

The following examples serve only to illustrate the invention and its practice. The examples are not to be construed as limitations on the scope or spirit of the invention.

### EXAMPLE 1

Preparation of Compound A and its
Form 1 Crystalline Potassium Salt

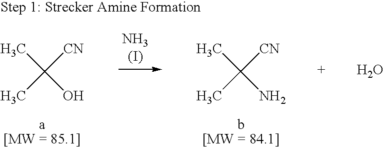

| | | | | | | density |
|---|---|---|---|---|---|---|
| Material | MW | Eq. | Moles | Mass | Volume | (g/mL) |
| acetone cyanohydrin | 85.1 | 1.0 | 129.3 | 11.0 kg | 11.8 L | 0.932 |
| (a) | | | | | | |
| MTBE | | 4.0 | | | 44 L | |
| ammonia (g) | 17.03 | 1.5 | 193.9 | 3.30 kg | 4.9 L | 0.674 |

Acetone cyanohydrin (11.5 kg, 12.3 L) was charged to a 5-gallon autoclave and the vessel placed under 5 psi nitrogen pressure. The autoclave was cooled to 10° C., and ammonia gas (˜3.44 kg), pressurized to 30 psi, was fed into the vessel until the reaction reached complete conversion as determined by GC assay (less than 0.5% a). The resulting suspension was transferred to a polyjug and the autoclave rinsed with MTBE (approximately 17 L). The reaction mixture and rinse were then charged to a 100-L extractor followed by MTBE (15 L), the mixture agitated, and the layers carefully separated. The aqueous layer was back-extracted with MTBE (5 L) and the layers carefully separated. The organic layers were combined and charged to a 100 L flask, equipped with a batch concentrator, through an in-line filter, and the batch was concentrated (15-20° C., low vacuum) to about 20 L to remove any excess ammonia. The aminonitrile was obtained in 97% assay yield (11.1 kg) by NMR as a solution in MTBE.

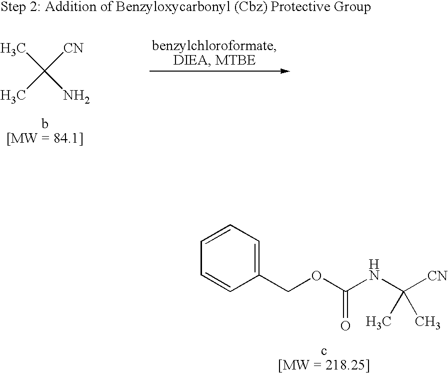

| Material | MW | Eq. | Moles | Mass | Volume |
|---|---|---|---|---|---|
| aminonitrile (b) | 84.1 | | 52.85 | 4.44 assay kg | |
| benzylchloroformate | 170.6 | 1.2 | 63.4 | 10.8 kg | |
| DIEA | 129.25 | 1.3 | 68.7 | 8.88 | |
| MTBE | | | | | 62.5 L |

To a visually clean 100-L flask containing a 5-L addition funnel, thermocouple and nitrogen inlet was charged a 59 wt. % solution of cyanoamine b in MTBE (4.44 assay kg). The solution was further diluted with MTBE (62.5 L) to bring the concentration to approximately 15 mL/g. Benzylchloroformate (1.20 equiv, 10.42 kg, 61.10 mol) was then charged in over 15 minutes via the addition funnel at such a rate as to maintain the batch temperature below 35° C. DIEA (1.3 equiv, 8.88 kg, 68.70 mol) was then added over 1.5 hours to the yellow slurry while maintaining the batch temperature below 35° C. The slurry became slightly more soluble as DIEA was added but two phases were observed when stirring was stopped. The reaction mixture was aged for 16 hours at 20-25° C., after which DI water (20 L, 4.5 mL/g) was charged into the batch. The batch was then transferred to a 100-L extractor and the phases were separated. The organic layer was then washed with 3×10 L of water and then 15 L of brine. The organic layer was transferred via a 10 μm inline filter to a 100 L round bottom flask and subsequently solvent switched to 90:10 heptane:MTBE. Crystallization occurred during the solvent switch and the resulting white crystalline product was filtered and washed with 3×5 L of 90:10 heptane:MTBE. A total of 10.1 kg of product (88% yield) was obtained in greater than 99 HPLC A %. A total of 26.7 kg of product was obtained in 3 batches with an average isolated yield of 86%.

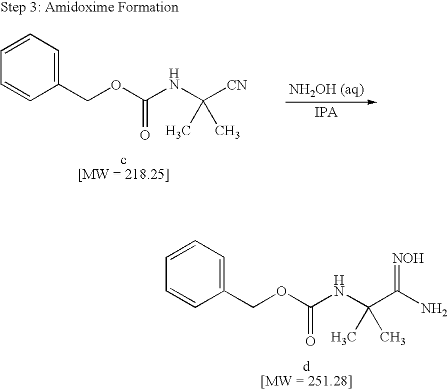

| Material | MW | Eq. | Mass | Volume |
|---|---|---|---|---|
| protected aminonitrile (c) | 218.25 | 1 | 15 g | |
| NH₂OH (50 wt. % in water) | | 1.2 | | 5.05 mL |
| IPA | | | | 40 mL + |
| | | | | 10 mL |
| n-heptane | | | | 40 mL + |
| | | | | 50 mL |

A solution of aminonitrile (15 g) in IPA (40 mL) was warmed to 60° C. with stirring and NH₂OH in water (5.05 mL) was added at this temperature over the course of 20 minutes. The clear mixture was then aged at 60° C. for 3 hours, wherein product began to crystallize out of solution at this temperature after 2 hours. The slurry was then cooled to 0-5° C. and n-heptane (40 mL) was added dropwise over 20 minutes. After stirring for 2 hours at 0°-5° C., the slurry was filtered and the cake was washed with a 20% IPA in heptane solution (60 mL), and then dried under vacuum with a nitrogen stream at room temperature to give pure amide oxime in 88% yield.

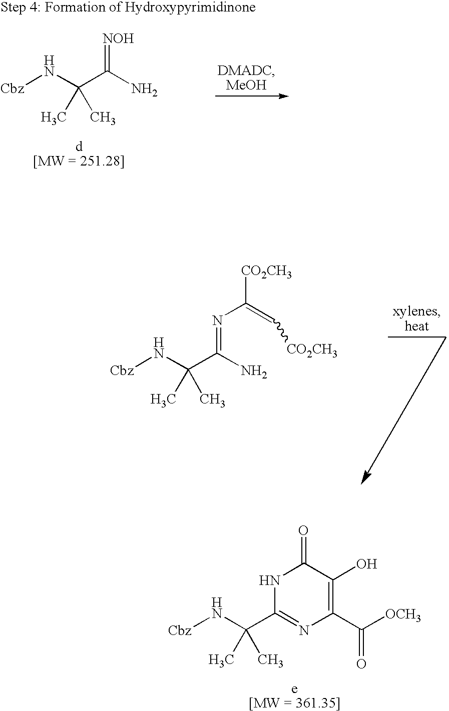

| | | | | | Density |
|---|---|---|---|---|---|
| Material | MW | Eq. | Mass | Volume | (g/mL) |
| amidoxime (d) | 251.28 | 1 | 2.9 kg | | |
| DMADC | 142.11 | 1.08 | 1.77 | | 1.16 |
| MeOH | | | | 12 L + 6 L | |
| xylenes | | | | 15 L | |
| MTBE | | | | 9 L | |

To a slurry of amidoxime (2.90 kg) in methanol (12 L) was added dimethyl acetylenedicarboxylate (1.77 kg) over 20 minutes. A slow exotherm ensued such that the temperature of the slurry increased from 20° C. to 30° C. over 15-20 minutes. After 1.5 hours, HPLC indicated greater than 95% conversion to the intermediate cis/trans adducts. The solvent was then switched to xylenes under reduced pressure (maximum temperature=50° C.), wherein 2 volumes [2×7.5 L] were added and reduced to a final volume of 7.5 L. The reaction mixture was then heated to 90° C. and kept at this temperature for 2 hours, while flushing the remaining MeOH out with a nitrogen sweep. The temperature was then increased in 10° C. increments over 3.5 hours to 125° C. and held at this temperature for 2 hours. The temperature was then finally increased to 135° C. for 5 hours. The reaction mixture was then cooled to 60° C. and MeOH (2.5 L) was added. After 30 minutes MTBE (9 L) was added slowly to build a seed bed. The batch was then cooled to 0° C. for 14 hours, and then further cooled to −5° C. and aged 1 hour before filtration. The solids were displacement washed with 10% MeOH/MTBE (6 L then 4 L; pre-chilled to 0° C.) and dried on the filter pot under a nitrogen sweep to afford 2.17 kg (51.7% corrected yield; 99.5 wt %).

HPLC method: Column: Zorbax C-8 4.6 mm×250 mm; 40% ACN/60% 0.1% H₃PO₄ to 90% ACN/10% 0.1% H₃PO₄ over 12 minutes, hold 3 minutes then back to 40% ACN over 1 minute. Retention times: amidoxime d-2.4 minutes, DMAD-6.7 minutes, intermediate adducts—8.4 and 8.6 minutes (8.4 minute peak cyclizes faster), product e-5.26 minutes, xylenes—several peaks around 10.4-10.7 minutes.

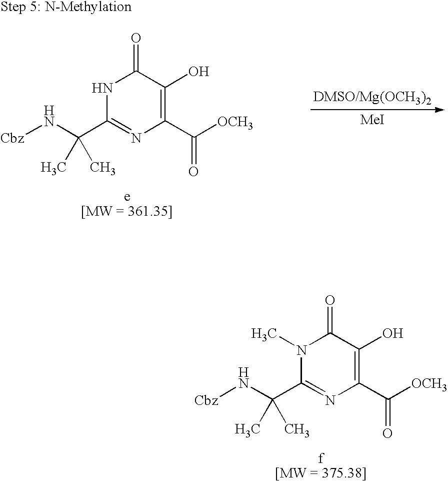

| Material | MW | Eq. | Mass | Volume |
|---|---|---|---|---|
| pyrimidine diol (e) | 361.35 | 1 | 2 kg | |
| Mg(OMe)₂, 8 wt. % in MeOH | | 2 | 11.95 kg | 13.4 L |
| MeI | | 4 | 3.14 kg | 1.38 L |
| DMSO | | | | 16 L |
| 2 M HCl | | | | 20 L |
| MeOH | | | | 14 L |
| Na bisulfite 5 wt. % in water | | | | 2 L |
| water | | | | 60 L |

To a solution of the pyrimidine diol e (2 kg) in DMSO (16 L) was added a solution of Mg(OMe)₂ in MeOH (11.95 kg), after which excess MeOH was evaporated under vacuum (30 mm Hg) at 40° C. for 30 minutes. The mixture was then cooled down to 20° C., after which MeI (1.38 L) was added and the mixture stirred at 20-25° C. for 2 hours, and then at 60° C. for 5 hours under pressure in a closed flask. HPLC showed that the reaction was complete. The mixture was then cooled to 20° C., after which MeOH (14 L) was added, followed by the slow addition of 2 M HCl (20 L) [exotherm] over 60 minutes. Sodium bisulfite (5 wt. %, 2 L) was then added to quench excess I₂, with the solution turning white. Water (40 L) was then added over 40 minutes and the slurry stirred for 40 minutes in an ice bath, and then filtered. The filter cake was washed first with water (20 L) and then with MTBE:MeOH 9/1 (30 L) to remove O-methylated by-product. HPLC showed less than 0.5 A % O-methylated product after washing. The solid was dried overnight at room temperature under vacuum with an N₂ stream to give 1.49 kg of N-methylpyrimidone (70% yield, corrected for purity of starting material and product).

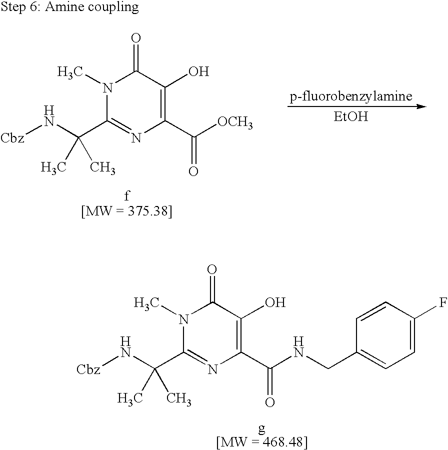

| Material | MW | Eq. | Mass | Volume |
|---|---|---|---|---|
| N-methylpyrimidinone (f) | 375.38 | 1 | 1.4 kg | |
| 4-fluorobenzylamine | 125.15 | 2.2 | 1.05 kg | |
| EtOH | | | | 14 L |
| water | | | | 14 L |
| acetic acid | | | | 0.55 L |

To a slurry of N-methylated pyrimidinone f (1.4 kg) in EtOH (14 L) at 4° C. was slowly added 4-fluorobenzylamine (1.05 kg) over 15 minutes, wherein an exotherm to 9° C. was observed during addition of the first 1 mole equivalent of the amine. The slurry became very thick and vigorous stirring was required. The reaction was warmed to 72° C. over 2 hours and maintained at this temperature for 1 hour and 45 minutes. The solution became extremely viscous at 45° C. where a small exotherm was observed to 50° C., after which the slurry slowly freed up and became homogeneous after 1 hour at 72° C. An HPLC sample assay (HPLC method was similar to that employed in Step 4 above) at the end of the reaction showed less than 0.5 A % N-methylated pyrimidinone. The reaction was then cooled to 60° C. and acetic acid (0.55 L) was added over 30 minutes, followed by the addition of water (6.7 L) over 30 min and then the addition of seed (3.0 g) to initiate crystallization. After 30 min at 60° C., more water (7.3 L) was added over 30 minutes and the reaction mixture allowed to cool to ambient temperature overnight. After 13 hours, the temperature was at 20° C., at which point the reaction mixture was filtered and the slurry washed with 50% water/EtOH (2×4 L). The solids were dried on the filter pot under vacuum/N₂ flow to a constant weight to afford a white solid product (1.59 kg; 90% corrected yield; 99% LCWP and 99.7% LCAP as determined by HPLCmethod similar to that employed in Step 4 above.)

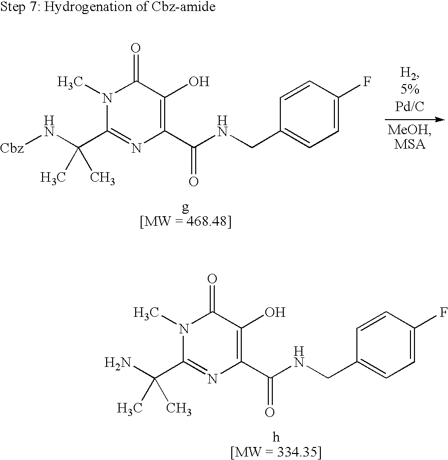

| Material | MW | mmoles | Mass | Volume |
|---|---|---|---|---|
| CBz amide (g) | 468.48 | 21.33 | 10 g | |
| MeOH | | | | 80 mL |
| 5% Pd/C (50% wet) | | | 0.15 g | |
| MSA | 96.1 | 22.4 | | 1.45 mL |
| water | | | | 8 mL |
| cake wash (4:1 MeOH:H₂O | | | | 20 mL |
| 1 N NaOH | | 22.4 | | 22.4 mL |
| final cake wash (water) | | | | 30 mL |

A stainless steel hydrogenation vessel was preconditioned with MeOH, Pd/C catalyst and MSA under the reaction conditions described below. Cbz-amide g (10 g) was then slurried in MeOH (80 mL) in the preconditioned vessel. MSA (1.45 mL) was added to the slurry in one portion at room temperature. 5% Pd/C (0.15 g, 50% wet) was also added to the hydrogenation vessel. Hydrogen was charged to the vessel in three successive vacuum/hydrogen purge cycles, after which the mixture was hydrogenated at 40 psig for 3-4 hour at 50° C. Following hydrogenation, water (8 mL) was added to the reaction mixture, the mixture was stirred, and the catalyst was filtered and washed with 4:1 MeOH:water (20 mL). The pH of combined filtrates was adjusted to pH 7 to 8.0 by slow addition of 1 N NaOH (22.4 mL), which precipitated a solid. The slurry was stirred at 0-5° C. for 4 hours and the solid filtered, washed with water (30 mL), collected and dried in vacuo at 50° C. The product amine (as hydrate) was obtained as a white crystalline solid (7.7 g) in 96% yield (corrected for KF), 89% LCWP, 99.8% LCAP, KF=11 wt. %

HPLC Method A (product assay): column: 25 cm×4.6 mm Zorbax RX-C8; mobile phase: A=0.1% H₃PO₄, B=CH₃CN, 0 minutes (80% A/20% B), 20 minutes (20% A/80% B), 25 minutes (20% A/80% B); flow: 1.0 mL/minute; wavelength: 210 nm; column temperature: 40° C.; retention times: des-fluoroamine byproduct—5.5 min, amine product—5.85 minutes, toluene—16.5 minutes, Cbz-amide—16.82 minutes.

HPLC Method B (product purity): column: 25 cm×4.6 mm YMC-basic; mobile phase: A=25 mmol KH₂PO₄ adjusted to pH=6.1, B=CH₃CN, 0 minutes (90% A/10% B), 30 minutes (30% A/70% B), 35 minutes (30% A/70% B); flow: 1 mL/minute; wavelength: 210 nm; column temperature: 30° C.; retention times: des-fluoroamine—9.1 minutes, amine—10.1 minutes, toluene—24.2 minutes, Cbz amide—25.7 minutes.

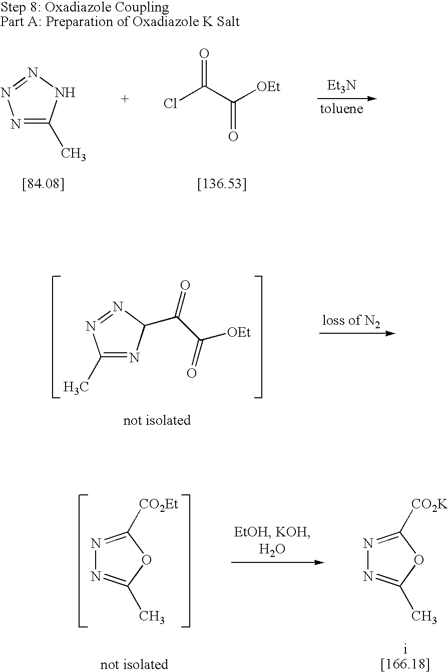

| Material | Eq. | Mole | Mass | Volume | Density |
|---|---|---|---|---|---|
| 5-methyltetrazole | 1.0 | 28.54 | 2.5 kg | | |
| (96 wt. %) | | | (2.4 kg) | | |
| ethyloxalyl chloride | 0.97 | 27.7 | 3.78 kg | 3.1 L | 1.22 |
| triethylamine | 0.99 | 28.3 | 2.86 kg | 4 L | 0.72 |
| toluene | | | | 67 L | |
| EtOH (punctilious) | | | | 61 L | |
| MTBE | | | | 15 L | |
| KOH aq. *20 wt. %) | | | | 8 L | |
| 10% brine | | | | 5 L | |

Ethyl oxalylchloride (3.78 kg) was slowly added to a mixture of 5-methyltetrazole (2.50 kg), triethylamine (2.86 kg) in toluene (32 L) at 0° C. at such a rate that the temperature stays below 5° C. The resulting slurry was stirred for 1 hour at 0-5° C. then heated to 60-65° C. over 1 hour (N₂ gas evolution). The slurry was aged at 60-65° C. for 1 hour and then cooled down to 20-25° C. The triethylamine/HCl salt was filtered off. The solid was washed with 27 L of toluene. The combined filtrates were washed with 5 L of 10% brine, then solvent switched to ethanol (reduced to 8-L, then 17 L of EtOH was added, then concentrated down to 8 L, then 33 liters of EtOH were added to adjust final volume of 41 L). The ethanol solution was cooled to 10° C. and KOH aq. (8.0 L) was added over 30 minutes, and the resulting thick slurry was then stirred for 40 minutes at room temperature while the oxadiazole K salt crystallized out. The solid was filtered off, washed with 11 L of EtOH and finally with 15 L of MTBE. The solid was dried overnight under vacuum at 20° C. with a nitrogen stream to yield 4.48 kg (90.8%) of the K-salt i.

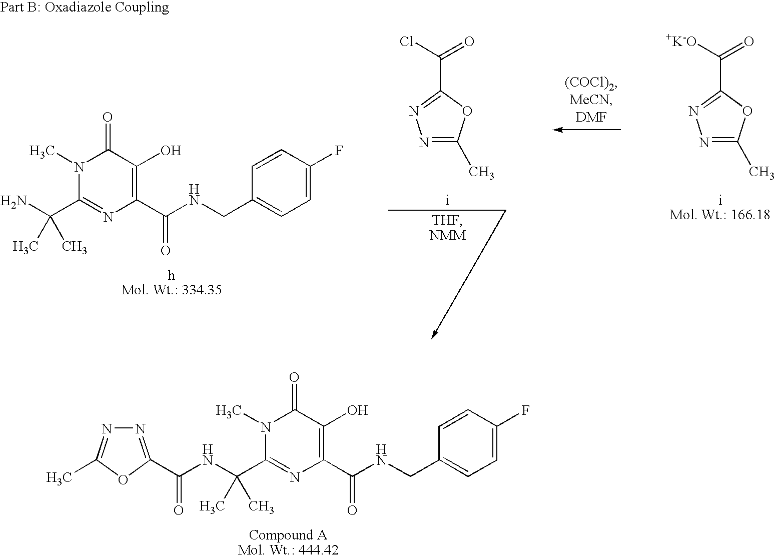

| Reagent | Mass | mL | Moles | Eq. |
|---|---|---|---|---|
| oxadiazole K salt i | 33.8 g (96.1 wt %) | | 0.20 | 2.2 |
| MeCN | | 280 mL | | |
| DMF | 0.33 | | | |
| oxalyl chloride | 23.7 g | 16.3 mL | 0.19 | 2.1 |
| free amine h | 30 g (99 wt %) | | 0.089 | 1 |
| THF | | 821 mL | | |
| NMM | 21.56 g | 23.4 mL | 0.21 | 2.4 |
| _{κ}OH (17% in H₂O) | 173 g | 150 mL | 0.53 | 5.9 |
| HCl (2 N) | | 300 mL | | |
| IPA | | 450 mL | | |
| water | | 550 mL | | |

A 500 mL round bottom flask was charged with oxadiazole K salt i (33.8 g) followed by MeCN (280 mL) and DMF (0.33 mL) with strong stirring. The resulting slurry was then cooled down to 0-5° C. and oxalyl chloride (23.7 g) was added over the course of 20 minutes in order to maintain the internal temperature at less than 5° C. The resulting acyl chloride-containing slurry was then aged for 1 hour.

To a 2 L round bottom flask free amine h (30 g) was added followed by THF (821 mL). The resulting slurry was cooled down to 0-5° C., after which NMM (21.56 g) was added and the slurry so obtained was stirred for 10 minutes at the cold temperature. The previously prepared acyl chloride-containing slurry was added slowly to the free amine slurry over the course of 20 minutes such that the temperature did not exceed 5° C. The slurry was then aged for 1.5 hours at 0-5° C. At this time HPLC showed no more amine b (<0.5% LCAP, 100% conversion). The reaction mixture was then quenched with KOH (17% in water) (150 mL) which was added over the course of 10 minutes. The resulting yellow slurry was then stirred for an additional hour at temperatures less than 10° C. The yellow slurry was then acidified to pH 3-4 with HCl (2N) (300 mL). To the resulting red wine colored solution, IPA (450 mL) was added. The low boiling point organic solvents were then evaporated under reduced pressure (40 torr) at room temperature to a final solution volume of 650 mL, at which volume crystalline Compound A began to precipitate. Water (350 mL) was then added to this new slurry over the course of 1 hour. The slurry was then cooled to 0-10° C. and aged for 2 hours. The aged slurry was filtered and the solid obtained was washed twice with water (100 mL each). The solid so obtained was dried overnight under vacuum and nitrogen stream to give 38.3 g of Compound A (95% yield).

### Step 9: Formation of a Crystalline Potassium Salt of Compound A

Compound A (400 g) was dissolved in 4 liters of 60:40 ethanol:acetonitrile at 45° C. to provide a solution of Compound A with a concentration of 95 g/L. Ethanol (1201 g) was added to 300 g of a 24 wt. % solution of potassium ethoxide in ethanol to obtain a 4.8 wt % solution of KOEt in ethanol. A seed bed was prepared by adding Form 1 crystalline potassium salt of Compound A (78 g) to 1.08 liters of 70:30 ethanol:aceontitrile. The seed bed was wet milled using an Ultra Turrax IKA T-50 mixer for 45 minutes at 10,000 rpm, reaching ˜50,000 particle counts (1-500 um) and a mean particle size of 10 um as determined with a Lasentec FBRM Model S400 particle size analyzer.

The seed slurry (1.16 liters) was charged to a crystallizer with a jacket temperature set to 35° C. The solution of Compound A at 45° C. was then charged to the seed slurry in the crystallizer. While agitating the Compound A solution-seed slurry at 250 rpm, the KOEt solution was charged to the crystallizer above the surface of the solution-seed slurry at a constant rate of 4.7 mL/minutes over 6 hours and 40 minutes. The crystallizer jacket temperature was set to 35° C. for the first 6 hours and then changed to 20° C. while the remaining ˜9% of ethoxide was charged over the last 40 minutes. The batch was aged at 20° C. for 30 minutes and filtered, and the resulting filter cake was washed with 2.8 L of ethanol. The washed cake was then blown with nitrogen for 1 hour and transferred to a vacuum oven and dried overnight at 45° C. to afford the title salt.

### EXAMPLE 2

### Form 1 Crystalline Potassium Salt of Compound A

### Part A: Preparation

Ethanol (147 mL), water (147 mL), and Compound A (97.9 g assay by HPLC) were charged to a 1 L round bottom flask equipped with mechanical stirrer, addition funnel, nitrogen inlet (i.e., run conducted under nitrogen), and a thermocouple. Aqueous KOH (45% w/w, 0.98 eq., 18.5 mL, 216 mmoles) was added to the suspension over 10 minutes at 21° C. The resulting suspension was agitated for 0.5 hour resulting in the dissolution of a majority of the solids, after which the batch was filtered through a 1 μm filter directly into a 5 L round bottom flask equipped with mechanical stirrer, addition funnel, nitrogen inlet, and thermocouple. The 1 L flask was rinsed with 1:1 (v/v) water/EtOH (48 mL) and the rinse was filtered into the 5 L crystallization vessel. The filtered solution was seeded with crystalline Form 1 Compound A K salt (200 mg) at room temperature and then aged for 1 hour to build a good seed bed, after which the suspension was diluted with EtOH (1.57 L) at 20° C. over 1.5 hour. The batch was then cooled to about 4° C. and aged until the concentration of Compound A in the mother liquor was measured to be 4.7 g/L. The batch was filtered, the crystallization vessel rinsed with 50 mL EtOH into the filter, the cake washed with EtOH (4×100 mL), and then dried under vacuum and a nitrogen tent until the amount of EtOH present by NMR was about 0.4 mol % relative to the potassium salt. The potassium salt of Compound A was obtained in 88% yield (91.5 g assay by HPLC, 99 area % by HPLC analysis).

### Part B: Characterization

An XRPD pattern of a K salt prepared in the manner described in Part A was generated on a Philips Analytical X'Pert Pro X-ray powder diffractometer using a continuous scan from 2.5 to 40 degrees 2Θ over about 12 minutes (i.e., 0.02° step size with 40 seconds/step), 2 RPS stage rotation, and a gonio scan axis. Copper K-Alpha 1 (K_{α1}) and K-Alpha 2 (K_{α2}) radiation was used as the source. The experiment was run under ambient conditions. 2Θ values and the corresponding d-spacings include the following:

| Peak No. | d-spacing (Å) | 2 Theta |
|---|---|---|
| 1 | 14.9 | 5.9 |
| 2 | 7.1 | 12.5 |
| 3 | 4.4 | 20.0 |
| 4 | 4.3 | 20.6 |
| 5 | 3.5 | 25.6 |

A K salt prepared in the manner described in Part A was also analyzed by a TA Instruments DSC 2910 differential scanning calorimeter at a heating rate of 10° C./min from room temperature to 350° C. in a crimped pinhole aluminum pan in a nitrogen atmosphere. The DSC curve, shown in FIG. 2, exhibited a single, sharp endotherm with a peak temperature of about 279° C. and an associated heat of fusion of about 230.0 J/gm. The endotherm is believed to be due to melting.

A thermogravimetric analysis was performed with a Perkin-Elmer Model TGA 7 under nitrogen at a heating rate of 10° C./min from room temperature to about 350° C. The TG curve showed a 0.3% weight loss during heating to 250° C.

Hygroscopicity data was obtained on a VTI Symmetrical Vapor Sorption Analyzer Model SGA-1. Data was collected at room temperature from 5-95% relative humidity and back, 5% relative humidity change per step. Equilibrium conditions were 0.01 weight percent change in 5 minutes with a maximum equilibration time of 180 minutes. The data indicated that the material had a 1.8% weight increase when equilibrated at 95% RH at 25° C. When equilibrated back down to 5% RH, the material returned back to approximately its dry weight. An XRPD analysis of the material after the hygroscopicity experiment showed that the material had not changed phases.

K salt prepared as described in Part A was also assayed by HCl titration using a Brinkmann Metrohm 716 DMS Titrino. The assay results indicated the salt was a monopotassium salt.

### EXAMPLE 3

Preparation of Compressed Tablets Containing
Compound A Potassium Salt

| | | |
|---|---|---|
| | Amount per | Amt per batch |
| Ingredient | Tablet (mg) | (wt. percent) |

Intragranular ingredients:

| | | |
|---|---|---|
| Compound A K salt¹ | 108.6 | 21.72 |
| (on free phenol basis) | (100) | (20.0) |
| microcrystalline cellulose | 26.0 | 5.2 |
| (AVICEL PH-102) | | |
| basic butylated methacrylate copolymer | 50.0 | 10 |
| (Eudragit E PO) | | |
| magnesium stearate | 4.2 | 0.84 |
| aspartame | 5.6 | 1.12 |
| natural banana flavor | 2.8 | 0.56 |
| (WONF Durarome (501392 TD0991)) | | |
| monoammonium glycyrrhizinate | 2.8 | 0.56 |
| (MAGNASWEET 135) | | |

Extragranular ingredients:

| | | |
|---|---|---|
| mannitol | 249 | 49.8 |
| (Pearlitol SD 200) | | |
| magnesium stearate | 15 | 3.0 |
| crospovidone | 25 | 5.0 |
| aspartame | 5 | 1.0 |
| natural banana flavor | 5 | 1.0 |
| (WONF Durarome (501392 TD0991) | | |
| yellow ferric oxide | 1 | 0.2 |
| Total | 500 | 100 |

¹Form 1 crystalline monopotassium salt of Compound A; conversion factor = 1.086.

Compressed tablets containing 100 mg of Compound A on a free phenol basis and suitable for use as orally disintegrating tablets were prepared by blending all of the intragranular ingredients listed in the above table except the magnesium stearate in a PK V blender (2 quart capacity; available from Patterson-Kelly Co., East Stroudsberg, Pa.) at a blending speed of 25 rpm for 10 minutes, then adding the magnesium stearate (IG) and blending for an additional 10 minutes. The blended mixture was then roller compacted and milled using an Alexanderwerk WP 120 roller compactor with the following parameters: 40 mm rolls, 40 mm feed screw, 3 rpm roll speed, 24 rpm screw speed, 70 bar roll pressure, 65 rpm RFG (rotary fine granulator) speed, 2 mm gap. The resulting granules were then blended with two-thirds of the magnesium stearate (EG) in the PK V blender for 10 minutes at 25 rpm, after which all of the remaining EG ingredients were added including the remaining one-third of the magnesium stearate (EG) and the admixture blended for 10 minutes at 25 rpm. This final blend was then compressed using a Manesty Beta press with 14/32-inch standard concave plain round tooling with a force of 4 to 6 kN (kN=kilonewtons) to provide tablets with a hardness of 2 to 3 kiloponds, wherein the hardness was measured using a Key HT-300 hardness tester.

### EXAMPLE 4

Preparation of Compressed Tablets Containing
Compound A Potassium Salt

| | | |
|---|---|---|
| | Amount per | Amt per batch |
| Ingredient | Tablet (mg) | (wt. percent) |

Intragranular ingredients:

| | | |
|---|---|---|
| Compound A K salt¹ | 27.16 | 6.79 |
| (on free phenol basis) | 25 | (6.25) |
| microcrystalline cellulose | 92.16 | 23.04 |
| (AVICEL PH-102) | | |
| basic butylated methacrylate copolymer | 12.52 | 3.13 |
| (Eudragit E PO) | | |
| magnesium stearate | 3 | 0.75 |
| aspartame | 4 | 1 |
| natural banana flavor | 2 | 0.5 |
| (WONF Durarome (501392 TD0991)) | | |
| monoammonium glycyrrhizinate | 2 | 0.5 |
| (MAGNASWEET 135) | | |

Extragranular ingredients:

| | | |
|---|---|---|
| mannitol | 216.36 | 54.09 |
| (Pearlitol SD 200) | | |
| magnesium stearate | 12 | 3 |
| crospovidone | 20 | 5 |
| aspartame | 4 | 1 |
| natural banana flavor | 4 | 1 |
| (WONF Durarome (501392 TD0991) | | |
| yellow ferric oxide | 0.8 | 0.2 |
| Total | 500 | 100 |

¹Form 1 crystalline monopotassium salt of Compound A; conversion factor = 1.086.

Compressed tablets containing 25 mg of Compound A on a free phenol basis and suitable for use as orally disintegrating tablets were prepared using the procedure described in Example 3, except that the final blend was compressed using a Korsch PH106 press with 13/32-inch standard concave plain round tooling with a force of 4 to 6 kilonewtons to provide tablets with a hardness of 2 to 3 kiloponds.

### EXAMPLE 5

Preparation of Compressed Tablets Containing
Compound A Potassium Salt

| | Amount per | Amt per batch |
|---|---|---|
| Ingredient | Tablet (mg) | (wt. percent) |
| Compound A K salt¹ | 108.6 | 21.72 |
| (on free phenol basis) | (100) | (20.0) |
| microcrystalline cellulose | 100 | 20.0 |
| (AVICEL PH-102) | | |
| basic butylated methacrylate copolymer | 50 | 10 |
| (Eudragit E PO) | | |
| crospovidone | 10 | 2.0 |
| mannitol | 198.05 | 39.61 |
| (Pearlitol SD 200) | | |
| magnesium stearate | 8.75 | 1.75 |
| aspartame | 11.2 | 2.24 |
| artificial grape flavor | 7.8 | 1.56 |
| monoammonium glycyrrhizinate | 5.6 | 1.12 |
| (MAGNASWEET 135) | | |
| Total | 500 | 100 |

¹Form 1 crystalline monopotassium salt of Compound A; conversion factor = 1.086.

Compressed tablets containing 100 mg of Compound A on a free phenol basis and suitable for use as chewable tablets were prepared by blending all of the ingredients listed in the above table in a Turbula T2F mixer (available from Glenmills, Inc., Clifton, N.J.) for 10 minutes, and then roller compacting the blend into ribbons using a Vector TF-Mini roller compactor (available from Vector Corp.) at 2.5 rpm roll speed, 10 rpm feed screw speed, and 50 kg/cm² roll pressure. The ribbons were then milled using a small cone mill (Quadro Comil Model No. 193; available from Quadro Inc., Milburn, N.J.) at approximately 2000 rpm with a 40 G (=grated) screen and round impeller to form granules, which were then compressed using an Auto Carver press (Model No. 388.1DI0A00, available from Carver, Inc., Wabash, Ind.) with 14/32-inch standard concave round tooling with a force of 10 to 12 kilonewtons to provide tablets having a hardness of 7 to 8 kiloponds.

Compressed tablets containing 25 mg and 50 mg of Compound A respectively on a free phenol basis were prepared in the same manner as the 100 mg tablets. The ingredients employed in the 25 mg and 50 mg tablets are as follows:

| | 25 mg tablets | 50 mg tablets |
|---|---|---|
| Ingredient | (wt. %) | (wt. %) |
| Compound A K salt¹ | 6.79 | 13.58 |
| (on free phenol basis) | (6.25) | (12.5) |
| microcrystalline cellulose | 20 | 20 |
| (AVICEL PH-102) | | |
| basic butylated methacrylate copolymer | 3.13 | 6.25 |
| (Eudragit E PO) | | |
| crospovidone | 2 | 2 |
| mannitol | 61.83 | 51.92 |
| (Pearlitol SD 200) | | |
| magnesium stearate | 1.75 | 1.75 |
| aspartame | 2 | 2 |
| artificial grape flavor | 1.5 | 1.5 |
| monoammonium glycyrrhizinate | 1 | 1 |
| (MAGNASWEET 135) | | |
| Total | 100 | 100 |

### EXAMPLE 6

### Dissolution Studies

The dissolution properties of the following types of tablets were tested:

A. 100 mg Compound A tablets prepared in the manner described in Example 3.

B. 25 mg Compound A tablets prepared in the manner described in Example 4.

The dissolution test was conducted in the following manner: Three A tablets and three B tablets were tested, wherein a single tablet was added to each of six USP Type II dissolution vessels containing 900 mL of 0.025 M sodium phosphate buffer (pH=7.5) as the dissolution medium. The temperature of the medium was controlled at 37° C. The medium was stirred for 1 hour at 50 rpm for the A tablets (100 mg Compound A) and for 1 hour at 75 rpm for the B tablets (25 mg Compound A). Samples (1.5 mL) were removed from the medium at 5, 10, 15, 20, 30, 45 and 60 minutes. Each sample was then analyzed via HPLC to determine the concentration of Compound A in the solution. The potassium salt of Compound A was employed as the reference standard, so a conversion factor of 0.9211 was used to obtain the results in terms of the free phenol form. The following table shows the results—the average percent of Compound A dissolved versus dissolution time.

HPLC: column=Merck KGaA Chromolith Performance RP-18e (100×4.6 mm); mobile phase=38:62 (v:v) of ACN: 0.01M K phosphate (pH=3.0); flow rate=5.0 mL/minute; column temperature=40° C.; injection volume=10 μL; detection wavelength=303 nm; run time=1 minute.

Dissolution Results

| Time | Tablet A | Tablet B |
|---|---|---|
| (minutes) | (% dissolved) | (% dissolved) |
| 5 | 85 (4.0) | 94 (3.0) |
| 10 | 96 (6.5) | 97 (2.5) |
| 15 | 99 (7.1) | 97 (2.0) |
| 20 | 99 (7.6) | 97 (1.5) |
| 30 | 99 (7.6) | 97 (1.5) |
| 45 | 100 (8.1) | 97 (1.5) |
| 60 | 100 (8.2) | 97 (1.5) |

The entries for percentage dissolved are the means for 3 tablets, with the standard deviation shown in parentheses.

### EXAMPLE 7

### Disintegration Studies

The disintegration time of each of Tablets A and B (see Example 6) was measured in accordance with the USP XXIV disintegration test procedure using a VanKel standard disintegration tester. The tests were conducted as follows for each of Tablets A and B: One tablet was placed in each of six tubes of the basket rack assembly and the tester was operated using deionized water at 37° C.(±2° C.) as the immersion medium, wherein the tablets were repeatedly immersed in the water at a rate of 30 strokes per minute until the tablets completely disintegrated. A tablet is defined as completely disintegrated when any residue of the tablet remaining on the tester's screen is a soft mass having no palpably firm core. The individual disintegration times of the 6 tablets were recorded. The disintegration time reported herein for the tablet is the average of the 6 measured times. The results are as follows:

Disintegration Time (seconds)

| Tablet | Run 1 | Run 2 | Run 3 |
|---|---|---|---|
| A | 18 | 19 | 20 |
| B | 17 | 18 | 17 |

### EXAMPLE 8

### Taste Test

A set of tablets containing 100 mg of Compound A (free phenol basis) (the “test” tablets) is prepared in the manner described in Example 3. A second set of 100 mg tablets (the “control” tablets) is prepared in the manner described in Example 3, except that the tablets contain no Eudragit E PO and instead have 22 wt. % of Avicel PH-102. The two sets of tablets are evaluated in a blind taste test by a panel consisting of 5 healthy adult male volunteers. Each member of the panel evaluates a test tablet and a control tablet. The panel member places a tablet in his mouth and allows the tablet to disintegrate completely without biting the tablet or drinking water. The time required for complete disintegration is measured. After complete disintegration, the panel member holds the disintegrated tablet in his mouth for thirty seconds and then rinses his mouth without ingesting the disintegrated tablet. The panel member then judges the palatability of the disintegrated tablet, evaluating its sweetness (from much too sweet to needs a lot more sweetness), bitterness (from not at all bitter to extremely bitter), mouth taste (from very pleasant to very unpleasant), and after taste (from very pleasant to very unpleasant). The panel member subsequently repeats the taste test with the other tablet.

A similar test can be conducted with tablets containing 25 mg of Compound A (free phenol basis) prepared in the manner described in Example 4.

### EXAMPLE 9

### Taste Test

A set of tablets containing 100 mg of Compound A (free phenol basis) (the “test” tablets) is prepared in the manner described in Example 5. A second set of 100 mg tablets (the “control” tablets) is prepared in the manner described in Example 5, except that the tablets contain no Eudragit E PO and instead have 22 wt. % of Avicel PH-102. The two sets of tablets are evaluated in a blind taste test by a panel consisting of 5 healthy adult male volunteers. Each member of the panel evaluates a test tablet and a control tablet. The panel member places a tablet in his mouth, completely chews the tablet without drinking any water, holds the chewed tablet in his mounth for thirty seconds and then rinses his mouth without ingesting the chewed tablet. The panel member then judges the palatability of the chewed tablet, evaluating its sweetness (from much too sweet to needs a lot more sweetness), bitterness (from not at all bitter to extremely bitter), mouth taste (from very pleasant to very unpleasant), and after taste (from very pleasant to very unpleasant). The panel member subsequently repeats the taste test with the other tablet.

A similar test can be conducted with tablets containing 25 mg or 50 mg of Compound A (free phenol basis) prepared in the manner described in Example 5.

While the foregoing specification teaches the principles of the present invention, with examples provided for the purpose of illustration, the practice of the invention encompasses all of the usual variations, adaptations and/or modifications that come within the scope of the following claims.

What is claimed is:

1. A pharmaceutical oral dosage form which is a tablet or granules, wherein the dosage form comprises:
(a) an effective amount of a carboxamide compound or a pharmaceutically acceptable salt thereof, wherein the carboxamide compound is (i) a 1-alkyl-5-hydroxy-6-oxo-1,6-dihydropyrimidine-4-carboxamide compound, (ii) a 3-hydroxy-4-oxo-6,7,8,9-tetrahydro-4H-pyrido[1,2-a]pyrimidine-2-carboxamide compound, or (iii) a 3-hydroxy-4-oxo-4,6,7,8,9,10-hexahydropyrimido[1,2-a]azepine-2-carboxamide compound;
(b) a taste-masking polymer; and
(c) a superdisintegrant.

2. A pharmaceutical oral dosage form according to claim 1, wherein:
(b) the taste-masking polymer comprises a cellulose polymer, a vinyl carboxylate-alkylene glycol copolymer, an acrylic polymer, a methacrylic polymer, or an acrylic-methacrylic copolymer; and
(c) the superdisintegrant comprises croscarmellose sodium, crospovidone, or sodium starch glycolate.

3. A pharmaceutical oral dosage form according to claim 2, wherein the taste-masking polymer comprises an aminoalkyl methacrylate copolymer.

4. A pharmaceutical oral dosage form according to claim 3, wherein the taste-masking polymer comprises Eudragit® E100 or Eudragit® PO.

5. A pharmaceutical oral dosage form according to claim 1, which further comprises: (d) a compression aid; (e) a water soluble filler; and (f) a lubricant.

6. A pharmaceutical oral dosage form according to claim 5, wherein:
(d) the compression aid comprises lactose, sucrose, anhydrous dibasic calcium phosphate, dibasic calcium phosphate dihydrate, tribasic calcium phosphate, calcium sulfate, carboxymethylcellulose calcium, microcrystalline cellulose, or powdered cellulose;
(e) the water soluble filler comprises mannitol, glucose, dextrose, or sucrose; and
(f) the lubricant comprises a metal stearate, a metal stearyl fumarate, or stearic acid.

7. A pharmaceutical oral dosage form according to claim 5, which further comprises (g) a sweetening agent, (h) a taste modifier, and optionally (i) a flavoring agent.

8. A pharmaceutical oral dosage form according to claim 5, wherein the dosage form is prepared by:
(A) a process which comprises (i) dry blending the carboxamide compound or its pharmaceutically acceptable salt, the taste-masking polymer, the superdisintegrant, the compression aid, the water soluble filler and the lubricant; (ii) granulating the dry blend to provide granules; and optionally (iii) compressing the granules to obtain a tablet; or
(B) a process which comprises (i) dry blending the carboxamide compound or its pharmaceutically acceptable salt, the taste-masking polymer, the compression aid, and a first portion or all of the lubricant; (ii) granulating the dry blend; (iii) mixing the granulated dry blend with the water soluble filler, the superdisintegrant, and the remaining portion (if any) of the lubricant; and compressing the mixture to obtain a tablet.

9. (canceled)

10. A pharmaceutical oral dosage form according to claim 1, wherein Compound I is Compound A or a pharmaceutically acceptable salt thereof, wherein Compound A is:

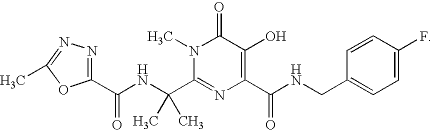

11. (canceled)

12. (canceled)

13. A pharmaceutical oral dosage form according to claim 1, wherein:
(a) the carboxamide compound is Compound A in the form of an alkali metal salt, wherein Compound A is 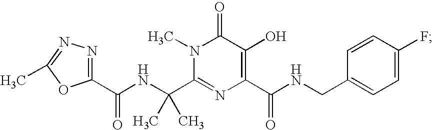
(b) the taste-masking polymer comprises an aminoalkyl methacrylate copolymer; and
(c) the superdisintegrant comprises crospovidone, croscarmellose sodium, or sodium starch glycolate.

14. A pharmaceutical oral dosage form according to claim 13, which further comprises:
(d) a compression aid which comprises lactose, sucrose, anhydrous dibasic calcium phosphate, dibasic calcium phosphate dihydrate, tribasic calcium phosphate, calcium sulfate, carboxymethylcellulose calcium, microcrystalline cellulose, or powdered cellulose;
(e) a water soluble filler which comprises mannitol;
(f) a lubricant comprises magnesium stearate, sodium stearyl fumarate, or stearic acid;
(g) optionally a sweetening agent which comprises aspartame, acesulfame potassium, sodium saccharin, or sucralose;
(h) optionally a taste modifier which comprises monoammonium glycyrrhizinate, sorbitol, maltose, maltodextrin, dextrose, or fructose; and
(i) optionally a flavoring agent.

15. A pharmaceutical oral dosage form according to claim 14, which is a tablet prepared by a process comprising:
(A) dry blending the alkali metal salt of Compound A, the aminoalkyl methacrylate copolymer, the superdisintegrant, the compression aid, the water soluble filler, the lubricant, the sweetening agent (if employed), the taste modifier (if employed), and the flavoring agent (if employed);
(B) granulating the dry blend to provide granules; and
(C) compressing the granules to obtain the tablet.

16. A pharmaceutical tablet according to claim 15, wherein:
(a) the alkali metal salt of Compound A is employed in an amount in a range of from about 1 to about 50 wt. % on a free phenol basis;
(b) the aminoalkyl methacrylate copolymer is employed in an amount in a range of from about 1 to about 25 wt. %;
(c) the superdisintegrant is employed in an amount in a range of from about 1 to about 15 wt. %;
(d) the compression aid is employed in an amount in a range of from about 5 to about 50 wt. %;
(e) the mannitol is employed in an amount in a range of from about 5 to about 75 wt. %;
(f) the lubricant is employed in an amount in a range of from about 0.1 to about 10 wt. %;
(g) the sweetening agent is employed in an amount in a range of from 0 to about 10 wt. %;
(h) the taste modifier is employed in an amount in a range of from 0 to about 2 wt. %; and
(i) the flavoring agent is employed in an amount in a range of from 0 to about 10 wt. %.

17. A pharmaceutical oral dosage form according to claim 14, which is a tablet prepared by a process comprising:
(A) dry blending the alkali metal salt of Compound A, the aminoalkyl methacrylate copolymer, the compression aid, a first portion of the lubricant, all or a first portion of the sweetening agent (if employed), all or a first portion of the taste modifier (if employed), and all or a first portion of the flavoring agent (if employed);
(B) granulating the dry blend to provide granules;
(C) mixing the granules with the superdisintegrant, the mannitol, the remaining portion of the lubricant, any remaining portion of the sweetening agent (if employed), any remaining portion of the taste modifier (if employed), and any remaining portion of the flavoring agent (if employed); and
(D) compressing the mixture to obtain the tablet.

18. A pharmaceutical tablet according to claim 17, wherein:
(a) the alkali metal salt of Compound A is employed in an amount in a range of from about 1 to about 50 wt. % on a free phenol basis;
(b) the aminoalkyl methacrylate copolymer is employed in an amount in a range of from about 1 to about 25 wt. %;
(c) the superdisintegrant is employed in an amount in a range of from about 1 to about 15 wt. %;
(d) the compression aid is employed in an amount in a range of from about 5 to about 50 wt. %;
(e) the mannitol is employed in an amount in a range of from about 5 to about 75 wt. %;
(f) the lubricant is employed in an amount in a range of from about 0.1 to about 5 wt. %;
(g) the sweetening agent is employed in an amount in a range of from 0 to about 5 wt. %;
(h) the taste modifier is employed in an amount in a range of from 0 to about 2 wt. %; and
(i) the flavoring agent is employed in an amount in a range of from 0 to about 5 wt. %.

19. A pharmaceutical tablet according to claim 18, wherein:
(a) the alkali metal salt of Compound A is a potassium salt of Compound A;
(b) the aminoalkyl methacrylate copolymer is Eudragit® E1100 or Eudragit® PO;
(c) the superdisintegrant is crospovidone;
(d) the compression aid is microcrystalline cellulose; and
(f) the lubricant is magnesium stearate.

20. A pharmaceutical tablet according to claim 19, wherein:
(a) the potassium salt of Compound A is employed in an amount in a range of from about 5 to about 25 wt. % on a free phenol basis;
(b) the Eudragit is employed in an amount in a range of from about 2 to about 15 wt. %;
(c) the crospovidone is employed in an amount in a range of from about 1 to about 5 wt. %;
(d) the microcrystalline cellulose is employed in an amount in a range of from about 5 to about 25 wt. %;
(e) the mannitol is employed in an amount in a range of from about 30 to about 60 wt. %;
(f) the magnesium stearate is employed in an amount in a range of from about 0.5 to about 2 wt. %;
(g) the sweetening agent is employed in an amount in a range of from 0 to about 2 wt. %;
(h) the taste modifier is each employed in an amount in a range of from 0 to about 1 wt. %; and
(i) the flavoring agent is employed in an amount in a range of from 0 to about 2 wt. %.

21. (canceled)

22. A pharmaceutical tablet according to claim 20, wherein the tablet formed by compressing the mixture has a hardness in a range of from about 2 to about 4 kiloponds.

23. A pharmaceutical tablet according to claim 22, wherein the amount of the potassium salt of Compound A is in a range of from about 20 to about 100 mg per tablet on a free phenol basis.

24. A method for the inhibition of HIV integrase, for the treatment or prophylaxis of HIV infection or for the treatment, prophylaxis or delay in the onset of AIDS in a subject in need thereof which comprises administering to the subject the pharmaceutical oral dosage form according to claim 1.

25. (canceled)
